SEQUENCE LISTING (16 sequences)

SEQ ID NO: 1 - protein, 252 aa, Plasmodium falciparum
PDCGVECKNETCTPKTVIYPDCGKNEKYEPPGDAKNTEINVINSGDKEGYIFEKLSEFCTNENNENGKNYEQWKCYYDNKKNNNKCKMEINIANSKLKNKITSFDEFFDFWVRKLLIDTIKWETELTYCINNTDVTDCNKCNKNCVCFDKWVKQKEDEWTNIMKLFTNKHDIPKKYYLNINDLFDSFFFQVIYKFNEGEAKWNELKENLKKQIASSKANNGTKDSEAAIKVLFNHIKEIATICKDNNTNEGC

SEQ ID NO: 2 - DNA, 759 bp, Plasmodium falciparum
  source 1..759: mol_type = genomic DNA
ccggattgcggcgtggaatgcaaaaacgaaacctgcaccccgaaaaccgtgatttatccggattgcggcaaaaacgaaaaatatgaaccgccgggcgatgcgaaaaacaccgaaattaacgtgattaacagcggcgataaagaaggctatatttttgaaaaactgagcgaattttgcaccaacgaaaacaacgaaaacggcaaaaactatgaacagtggaaatgctattatgataacaaaaaaaacaacaacaaatgcaaaatggaaattaacattgcgaacagcaaactgaaaaacaaaattaccagctttgatgaattttttgatttttgggtgcgcaaactgctgattgataccattaaatgggaaaccgaactgacctattgcattaacaacaccgatgtgaccgattgcaacaaatgcaacaaaaactgcgtgtgctttgataaatgggtgaaacagaaagaagatgaatggaccaacattatgaaactgtttaccaacaaacatgatattccgaaaaaatattatctgaacattaacgatctgtttgatagctttttttttcaggtgatttataaatttaacgaaggcgaagcgaaatggaacgaactgaaagaaaacctgaaaaaacagattgcgagcagcaaagcgaacaacggcaccaaagatagcgaagcggcgattaaagtgctgtttaaccatattaaagaaattgcgaccatttgcaaagataacaacaccaacgaaggctgctaa

SEQ ID NO: 3 - DNA, 1383 bp, Homo sapiens
  source 1..1383: mol_type = genomic DNA
atgtggcagctgaccagcctgctgctgtttgtggcgacctggggcattagcggcaccccggcgccgctggatagcgtgtttagcagcagcgaacgcgcgcatcaggtgctgcgcattcgcaaacgcgcgaacagctttctggaagaactgcgccatagcagcctggaacgcgaatgcattgaagaaatttgcgattttgaagaagcgaaagaaatttttcagaacgtggatgataccctggcgttttggagcaaacatgtggatggcgatcagtgcctggtgctgccgctggaacatccgtgcgcgagcctgtgctgcggccatggcacctgcattgatggcattggcagctttagctgcgattgccgcagcggctgggaaggccgcttttgccagcgcgaagtgagctttctgaactgcagcctggataacggcggctgcacccattattgcctggaagaagtgggctggcgccgctgcagctgcgcgccgggctataaactgggcgatgatctgctgcagtgccatccggcggtgaaatttccgtgcggccgcccgtggaaacgcatggaaaaaaaacgcagccatctgaaacgcgataccgaagatcaggaagatcaggtggatccgcgcctgattgatggcaaaatgacccgccgcggcgatagcccgtggcaggtggtgctgctggatagcaaaaaaaaactggcgtgcggcgcggtgctgattcatccgagctgggtgctgaccgcggcgcattgcatggatgaaagcaaaaaactgctggtgcgcctgggcgaatatgatctgcgccgctgggaaaaatgggaactggatctggatattaaagaagtgtttgtgcatccgaactatagcaaaagcaccaccgataacgatattgcgctgctgcatctggcgcagccggcgaccctgagccagaccattgtgccgatttgcctgccggatagcggcctggcggaacgcgaactgaaccaggcgggccaggaaaccctggtgaccggctggggctatcatagcagccgcgaaaaagaagcgaaacgcaaccgcacctttgtgctgaactttattaaaattccggtggtgccgcataacgaatgcagcgaagtgatgagcaacatggtgagcgaaaacatgctgtgcgcgggcattctgggcgatcgccaggatgcgtgcgaaggcgatagcggcggcccgatggtggcgagctttcatggcacctggtttctggtgggcctggtgagctggggcgaaggctgcggcctgctgcataactatggcgtgtataccaaagtgagccgctatctggattggattcatggccatattcgcgataaagaagcgccgcagaaaagctgggcgccg

SEQ ID NO: 4 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 5 - DNA, 1215 bp, Homo sapiens
  source 1..1215: mol_type = genomic DNA
atgtggcagctgaccagcctgctgctgtttgtggcgacctggggcattagcggcaccccggcgccgctggatagcgtgtttagcagcagcgaacgcgcgcatcaggtgctgcgcattcgcaaacgccatccgtgcgcgagcctgtgctgcggccatggcacctgcattgatggcattggcagctttagctgcgattgccgcagcggctgggaaggccgcttttgccagcgcgaagtgagctttctgaactgcagcctggataacggcggctgcacccattattgcctggaagaagtgggctggcgccgctgcagctgcgcgccgggctataaactgggcgatgatctgctgcagtgccatccggcggtgaaatttccgtgcggccgcccgtggaaacgcatggaaaaaaaacgcagccatctgaaacgcgataccgaagatcaggaagatcaggtggatccgcgcctgattgatggcaaaatgacccgccgcggcgatagcccgtggcaggtggtgctgctggatagcaaaaaaaaactggcgtgcggcgcggtgctgattcatccgagctgggtgctgaccgcggcgcattgcatggatgaaagcaaaaaactgctggtgcgcctgggcgaatatgatctgcgccgctgggaaaaatgggaactggatctggatattaaagaagtgtttgtgcatccgaactatagcaaaagcaccaccgataacgatattgcgctgctgcatctggcgcagccggcgaccctgagccagaccattgtgccgatttgcctgccggatagcggcctggcggaacgcgaactgaaccaggcgggccaggaaaccctggtgaccggctggggctatcatagcagccgcgaaaaagaagcgaaacgcaaccgcacctttgtgctgaactttattaaaattccggtggtgccgcataacgaatgcagcgaagtgatgagcaacatggtgagcgaaaacatgctgtgcgcgggcattctgggcgatcgccaggatgcgtgcgaaggcgatagcggcggcccgatggtggcgagctttcatggcacctggtttctggtgggcctggtgagctggggcgaaggctgcggcctgctgcataactatggcgtgtataccaaagtgagccgctatctggattggattcatggccatattcgcgataaagaagcgccgcagaaaagctgggcgccg

SEQ ID NO: 6 - protein, 405 aa, Homo sapiens
MWQLTSLLLFVATWGISGTPAPLDSVFSSSERAHQVLRIRKRHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 7 - protein, 643 aa, synthetic construct
  REGION 1..643: note = truncated human APC molecule fused to the PfEMP1 CIDR domain
MRLAVGALLVCAVLGLCLPDCGVECKNETCTPKTVIYPDCGKNEKYEPPGDAKNTEINVINSGDKEGYIFEKLSEFCTNENNENGKNYEQWKCYYDNKKNNNKCKMEINIANSKLKNKITSFDEFFDFWVRKLLIDTIKWETELTYCINNTDVTDCNKCNKNCVCFDKWVKQKEDEWTNIMKLFTNKHDIPKKYYLNINDLFDSFFFQVIYKFNEGEAKWNELKENLKKQIASSKANNGTKDSEAAIKVLFNHIKEIATICKDNNTNEGCGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 8 - DNA, 1932 bp, synthetic construct
  misc_feature 1..1932: note = truncated human APC molecule fused to the PfEMP1 CIDR domain
  source 1..1932: mol_type = other DNA
atgcgcctggcggtgggcgcgctgctggtgtgcgcggtgctgggcctgtgcctgccggattgcggcgtggaatgcaaaaacgaaacctgcaccccgaaaaccgtgatttatccggattgcggcaaaaacgaaaaatatgaaccgccgggcgatgcgaaaaacaccgaaattaacgtgattaacagcggcgataaagaaggctatatttttgaaaaactgagcgaattttgcaccaacgaaaacaacgaaaacggcaaaaactatgaacagtggaaatgctattatgataacaaaaaaaacaacaacaaatgcaaaatggaaattaacattgcgaacagcaaactgaaaaacaaaattaccagctttgatgaattttttgatttttgggtgcgcaaactgctgattgataccattaaatgggaaaccgaactgacctattgcattaacaacaccgatgtgaccgattgcaacaaatgcaacaaaaactgcgtgtgctttgataaatgggtgaaacagaaagaagatgaatggaccaacattatgaaactgtttaccaacaaacatgatattccgaaaaaatattatctgaacattaacgatctgtttgatagctttttttttcaggtgatttataaatttaacgaaggcgaagcgaaatggaacgaactgaaagaaaacctgaaaaaacagattgcgagcagcaaagcgaacaacggcaccaaagatagcgaagcggcgattaaagtgctgtttaaccatattaaagaaattgcgaccatttgcaaagataacaacaccaacgaaggctgcggcgatcagtgcctggtgctgccgctggaacatccgtgcgcgagcctgtgctgcggccatggcacctgcattgatggcattggcagctttagctgcgattgccgcagcggctgggaaggccgcttttgccagcgcgaagtgagctttctgaactgcagcctggataacggcggctgcacccattattgcctggaagaagtgggctggcgccgctgcagctgcgcgccgggctataaactgggcgatgatctgctgcagtgccatccggcggtgaaatttccgtgcggccgcccgtggaaacgcatggaaaaaaaacgcagccatctgaaacgcgataccgaagatcaggaagatcaggtggatccgcgcctgattgatggcaaaatgacccgccgcggcgatagcccgtggcaggtggtgctgctggatagcaaaaaaaaactggcgtgcggcgcggtgctgattcatccgagctgggtgctgaccgcggcgcattgcatggatgaaagcaaaaaactgctggtgcgcctgggcgaatatgatctgcgccgctgggaaaaatgggaactggatctggatattaaagaagtgtttgtgcatccgaactatagcaaaagcaccaccgataacgatattgcgctgctgcatctggcgcagccggcgaccctgagccagaccattgtgccgatttgcctgccggatagcggcctggcggaacgcgaactgaaccaggcgggccaggaaaccctggtgaccggctggggctatcatagcagccgcgaaaaagaagcgaaacgcaaccgcacctttgtgctgaactttattaaaattccggtggtgccgcataacgaatgcagcgaagtgatgagcaacatggtgagcgaaaacatgctgtgcgcgggcattctgggcgatcgccaggatgcgtgcgaaggcgatagcggcggcccgatggtggcgagctttcatggcacctggtttctggtgggcctggtgagctggggcgaaggctgcggcctgctgcataactatggcgtgtataccaaagtgagccgctatctggattggattcatggccatattcgcgataaagaagcgccgcagaaaagctgggcgccgtaa

SEQ ID NO: 9 - protein, 466 aa, Homo sapiens
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 10 - DNA, 1401 bp, Homo sapiens
  source 1..1401: mol_type = genomic DNA
atggtgagccaggcgctgcgcctgctgtgcctgctgctgggcctgcagggctgcctggcggcgggcggcgtggcgaaagcgagcggcggcgaaacccgcgatatgccgtggaaaccgggcccgcatcgcgtgtttgtgacccaggaagaagcgcatggcgtgctgcatcgccgccgccgcgcgaacgcgtttctggaagaactgcgcccgggcagcctggaacgcgaatgcaaagaagaacagtgcagctttgaagaagcgcgcgaaatttttaaagatgcggaacgcaccaaactgttttggattagctatagcgatggcgatcagtgcgcgagcagcccgtgccagaacggcggcagctgcaaagatcagctgcagagctatatttgcttttgcctgccggcgtttgaaggccgcaactgcgaaacccataaagatgatcagctgatttgcgtgaacgaaaacggcggctgcgaacagtattgcagcgatcataccggcaccaaacgcagctgccgctgccatgaaggctatagcctgctggcggatggcgtgagctgcaccccgaccgtggaatatccgtgcggcaaaattccgattctggaaaaacgcaacgcgagcaaaccgcagggccgcattgtgggcggcaaagtgtgcccgaaaggcgaatgcccgtggcaggtgctgctgctggtgaacggcgcgcagctgtgcggcggcaccctgattaacaccatttgggtggtgagcgcggcgcattgctttgataaaattaaaaactggcgcaacctgattgcggtgctgggcgaacatgatctgagcgaacatgatggcgatgaacagagccgccgcgtggcgcaggtgattattccgagcacctatgtgccgggcaccaccaaccatgatattgcgctgctgcgcctgcatcagccggtggtgctgaccgatcatgtggtgccgctgtgcctgccggaacgcacctttagcgaacgcaccctggcgtttgtgcgctttagcctggtgagcggctggggccagctgctggatcgcggcgcgaccgcgctggaactgatggtgctgaacgtgccgcgcctgatgacccaggattgcctgcagcagagccgcaaagtgggcgatagcccgaacattaccgaatatatgttttgcgcgggctatagcgatggcagcaaagatagctgcaaaggcgatagcggcggcccgcatgcgacccattatcgcggcacctggtatctgaccggcattgtgagctggggccagggctgcgcgaccgtgggccattttggcgtgtatacccgcgtgagccagtatattgaatggctgcagaaactgatgcgcagcgaaccgcgcccgggcgtgctgctgcgcgcgccgtttccgtaa

SEQ ID NO: 11 - protein, 631 aa, synthetic construct
  REGION 1..631: note = human Factor VIIa molecule fused to the PfEMP1 CIDR domain
MRLAVGALLVCAVLGLCLPDCGVECKNETCTPKTVIYPDCGKNEKYEPPGDAKNTEINVINSGDKEGYIFEKLSEFCTNENNENGKNYEQWKCYYDNKKNNNKCKMEINIANSKLKNKITSFDEFFDFWVRKLLIDTIKWETELTYCINNTDVTDCNKCNKNCVCFDKWVKQKEDEWTNIMKLFTNKHDIPKKYYLNINDLFDSFFFQVIYKFNEGEAKWNELKENLKKQIASSKANNGTKDSEAAIKVLFNHIKEIATICKDNNTNEGCDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 12 - DNA, 1896 bp, synthetic construct
  misc_feature 1..1896: note = human Factor VIIa molecule fused to the PfEMP1 CIDR domain
  source 1..1896: mol_type = other DNA
atgcgcctggcggtgggcgcgctgctggtgtgcgcggtgctgggcctgtgcctgccggattgcggcgtggaatgcaaaaacgaaacctgcaccccgaaaaccgtgatttatccggattgcggcaaaaacgaaaaatatgaaccgccgggcgatgcgaaaaacaccgaaattaacgtgattaacagcggcgataaagaaggctatatttttgaaaaactgagcgaattttgcaccaacgaaaacaacgaaaacggcaaaaactatgaacagtggaaatgctattatgataacaaaaaaaacaacaacaaatgcaaaatggaaattaacattgcgaacagcaaactgaaaaacaaaattaccagctttgatgaattttttgatttttgggtgcgcaaactgctgattgataccattaaatgggaaaccgaactgacctattgcattaacaacaccgatgtgaccgattgcaacaaatgcaacaaaaactgcgtgtgctttgataaatgggtgaaacagaaagaagatgaatggaccaacattatgaaactgtttaccaacaaacatgatattccgaaaaaatattatctgaacattaacgatctgtttgatagctttttttttcaggtgatttataaatttaacgaaggcgaagcgaaatggaacgaactgaaagaaaacctgaaaaaacagattgcgagcagcaaagcgaacaacggcaccaaagatagcgaagcggcgattaaagtgctgtttaaccatattaaagaaattgcgaccatttgcaaagataacaacaccaacgaaggctgcgatggcgatcagtgcgcgagcagcccgtgccagaacggcggcagctgcaaagatcagctgcagagctatatttgcttttgcctgccggcgtttgaaggccgcaactgcgaaacccataaagatgatcagctgatttgcgtgaacgaaaacggcggctgcgaacagtattgcagcgatcataccggcaccaaacgcagctgccgctgccatgaaggctatagcctgctggcggatggcgtgagctgcaccccgaccgtggaatatccgtgcggcaaaattccgattctggaaaaacgcaacgcgagcaaaccgcagggccgcattgtgggcggcaaagtgtgcccgaaaggcgaatgcccgtggcaggtgctgctgctggtgaacggcgcgcagctgtgcggcggcaccctgattaacaccatttgggtggtgagcgcggcgcattgctttgataaaattaaaaactggcgcaacctgattgcggtgctgggcgaacatgatctgagcgaacatgatggcgatgaacagagccgccgcgtggcgcaggtgattattccgagcacctatgtgccgggcaccaccaaccatgatattgcgctgctgcgcctgcatcagccggtggtgctgaccgatcatgtggtgccgctgtgcctgccggaacgcacctttagcgaacgcaccctggcgtttgtgcgctttagcctggtgagcggctggggccagctgctggatcgcggcgcgaccgcgctggaactgatggtgctgaacgtgccgcgcctgatgacccaggattgcctgcagcagagccgcaaagtgggcgatagcccgaacattaccgaatatatgttttgcgcgggctatagcgatggcagcaaagatagctgcaaaggcgatagcggcggcccgcatgcgacccattatcgcggcacctggtatctgaccggcattgtgagctggggccagggctgcgcgaccgtgggccattttggcgtgtatacccgcgtgagccagtatattgaatggctgcagaaactgatgcgcagcgaaccgcgcccgggcgtgctgctgcgcgcgccgtttccgtaa

SEQ ID NO: 13 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPQSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGQTATSEYQTFFNPQTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 14 - DNA, 1869 bp, Homo sapiens
  source 1..1869: mol_type = genomic DNA
atggcgcatgtgcgcggcctgcagctgccgggctgcctggcgctggcggcgctgtgcagcctggtgcatagccagcatgtgtttctggcgccgcagcaggcgcgcagcctgctgcagcgcgtgcgccgcgcgaacacctttctggaagaagtgcgcaaaggcaacctggaacgcgaatgcgtggaagaaacctgcagctatgaagaagcgtttgaagcgctggaaagcagcaccgcgaccgatgtgttttgggcgaaatataccgcgtgcgaaaccgcgcgcaccccgcgcgataaactggcggcgtgcctggaaggcaactgcgcggaaggcctgggcaccaactatcgcggccatgtgaacattacccgcagcggcattgaatgccagctgtggcgcagccgctatccgcataaaccggaaattaacagcaccacccatccgggcgcggatctgcaggaaaacttttgccgcaacccggatagcagcaccaccggcccgtggtgctataccaccgatccgaccgtgcgccgccaggaatgcagcattccggtgtgcggccaggatcaggtgaccgtggcgatgaccccgcagagcgaaggcagcagcgtgaacctgagcccgccgctggaacagtgcgtgccggatcgcggccagcagtatcagggccgcctggcggtgaccacccatggcctgccgtgcctggcgtgggcgagcgcgcaggcgaaagcgctgagcaaacatcaggattttaacagcgcggtgcagctggtggaaaacttttgccgcaacccggatggcgatgaagaaggcgtgtggtgctatgtggcgggcaaaccgggcgattttggctattgcgatctgaactattgcgaagaagcggtggaagaagaaaccggcgatggcctggatgaagatagcgatcgcgcgattgaaggccagaccgcgaccagcgaatatcagaccttttttaacccgcagacctttggcagcggcgaagcggattgcggcctgcgcccgctgtttgaaaaaaaaagcctggaagataaaaccgaacgcgaactgctggaaagctatattgatggccgcattgtggaaggcagcgatgcggaaattggcatgagcccgtggcaggtgatgctgtttcgcaaaagcccgcaggaactgctgtgcggcgcgagcctgattagcgatcgctgggtgctgaccgcggcgcattgcctgctgtatccgccgtgggataaaaactttaccgaaaacgatctgctggtgcgcattggcaaacatagccgcacccgctatgaacgcaacattgaaaaaattagcatgctggaaaaaatttatattcatccgcgctataactggcgcgaaaacctggatcgcgatattgcgctgatgaaactgaaaaaaccggtggcgtttagcgattatattcatccggtgtgcctgccggatcgcgaaaccgcggcgagcctgctgcaggcgggctataaaggccgcgtgaccggctggggcaacctgaaagaaacctggaccgcgaacgtgggcaaaggccagccgagcgtgctgcaggtggtgaacctgccgattgtggaacgcccggtgtgcaaagatagcacccgcattcgcattaccgataacatgttttgcgcgggctataaaccggatgaaggcaaacgcggcgatgcgtgcgaaggcgatagcggcggcccgtttgtgatgaaaagcccgtttaacaaccgctggtatcagatgggcattgtgagctggggcgaaggctgcgatcgcgatggcaaatatggcttttatacccatgtgtttcgcctgaaaaaatggattcagaaagtgattgatcagtttggcgaataa

SEQ ID NO: 15 - protein, 811 aa, synthetic construct
  REGION 1..811: note = human meizothrombin molecule fused to the PfEMP1 CIDR domain
MRLAVGALLVCAVLGLCLHHHHHHPDCGVECKNETCTPKTVIYPDCGKNEKYEPPGDAKNTEINVINSGDKEGYIFEKLSEFCTNENNENGKNYEQWKCYYDNKKNNNKCKMEINIANSKLKNKITSFDEFFDFWVRKLLIDTIKWETELTYCINNTDVTDCNKCNKNCVCFDKWVKQKEDEWTNIMKLFTNKHDIPKKYYLNINDLFDSFFFQVIYKFNEGEAKWNELKENLKKQIASSKANNGTKDSEAAIKVLFNHIKEIATICKDNNTNEGCTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPQSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGQTATSEYQTFFNPQTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 16 - DNA, 2436 bp, synthetic construct
  misc_feature 1..2436: note = human meizothrombin molecule fused to the PfEMP1 CIDR domain
  source 1..2436: mol_type = other DNA
atgcgcctggcggtgggcgcgctgctggtgtgcgcggtgctgggcctgtgcctgcatcatcatcatcatcatccggattgcggcgtggaatgcaaaaacgaaacctgcaccccgaaaaccgtgatttatccggattgcggcaaaaacgaaaaatatgaaccgccgggcgatgcgaaaaacaccgaaattaacgtgattaacagcggcgataaagaaggctatatttttgaaaaactgagcgaattttgcaccaacgaaaacaacgaaaacggcaaaaactatgaacagtggaaatgctattatgataacaaaaaaaacaacaacaaatgcaaaatggaaattaacattgcgaacagcaaactgaaaaacaaaattaccagctttgatgaattttttgatttttgggtgcgcaaactgctgattgataccattaaatgggaaaccgaactgacctattgcattaacaacaccgatgtgaccgattgcaacaaatgcaacaaaaactgcgtgtgctttgataaatgggtgaaacagaaagaagatgaatggaccaacattatgaaactgtttaccaacaaacatgatattccgaaaaaatattatctgaacattaacgatctgtttgatagctttttttttcaggtgatttataaatttaacgaaggcgaagcgaaatggaacgaactgaaagaaaacctgaaaaaacagattgcgagcagcaaagcgaacaacggcaccaaagatagcgaagcggcgattaaagtgctgtttaaccatattaaagaaattgcgaccatttgcaaagataacaacaccaacgaaggctgcaccgcgtgcgaaaccgcgcgcaccccgcgcgataaactggcggcgtgcctggaaggcaactgcgcggaaggcctgggcaccaactatcgcggccatgtgaacattacccgcagcggcattgaatgccagctgtggcgcagccgctatccgcataaaccggaaattaacagcaccacccatccgggcgcggatctgcaggaaaacttttgccgcaacccggatagcagcaccaccggcccgtggtgctataccaccgatccgaccgtgcgccgccaggaatgcagcattccggtgtgcggccaggatcaggtgaccgtggcgatgaccccgcagagcgaaggcagcagcgtgaacctgagcccgccgctggaacagtgcgtgccggatcgcggccagcagtatcagggccgcctggcggtgaccacccatggcctgccgtgcctggcgtgggcgagcgcgcaggcgaaagcgctgagcaaacatcaggattttaacagcgcggtgcagctggtggaaaacttttgccgcaacccggatggcgatgaagaaggcgtgtggtgctatgtggcgggcaaaccgggcgattttggctattgcgatctgaactattgcgaagaagcggtggaagaagaaaccggcgatggcctggatgaagatagcgatcgcgcgattgaaggccagaccgcgaccagcgaatatcagaccttttttaacccgcagacctttggcagcggcgaagcggattgcggcctgcgcccgctgtttgaaaaaaaaagcctggaagataaaaccgaacgcgaactgctggaaagctatattgatggccgcattgtggaaggcagcgatgcggaaattggcatgagcccgtggcaggtgatgctgtttcgcaaaagcccgcaggaactgctgtgcggcgcgagcctgattagcgatcgctgggtgctgaccgcggcgcattgcctgctgtatccgccgtgggataaaaactttaccgaaaacgatctgctggtgcgcattggcaaacatagccgcacccgctatgaacgcaacattgaaaaaattagcatgctggaaaaaatttatattcatccgcgctataactggcgcgaaaacctggatcgcgatattgcgctgatgaaactgaaaaaaccggtggcgtttagcgattatattcatccggtgtgcctgccggatcgcgaaaccgcggcgagcctgctgcaggcgggctataaaggccgcgtgaccggctggggcaacctgaaagaaacctggaccgcgaacgtgggcaaaggccagccgagcgtgctgcaggtggtgaacctgccgattgtggaacgcccggtgtgcaaagatagcacccgcattcgcattaccgataacatgttttgcgcgggctataaaccggatgaaggcaaacgcggcgatgcgtgcgaaggcgatagcggcggcccgtttgtgatgaaaagcccgtttaacaaccgctggtatcagatgggcattgtgagctggggcgaaggctgcgatcgcgatggcaaatatggcttttatacccatgtgtttcgcctgaaaaaatggattcagaaagtgattgatcagtttggcgaataa

(19) United States
(12) Patent Application Publication
(10) Pub. No.: US 2025/0066436 A1
(43) Pub. Date: Feb. 27, 2025

(54) A CONJUGATE FOR USE IN LOCALISING A MOLECULE TO THE VASCULAR ENDOTHELIUM

(71) Applicant: ROYAL COLLEGE OF SURGEONS IN IRELAND, Dublin (IE)

(72) Inventors: Roger PRESTON, Dublin (IE); Orla WILLIS FOX, Dublin (IE)

(21) Appl. No.: 18/723,022

(22) PCT Filed: Dec. 20, 2022
(86) PCT No.: PCT/EP2022/087001

§ 371 Date: Jun. 21, 2024

(30) Foreign Application Priority Data
Dec. 22, 2021 (EP) 21217130.0

(51) Int. Cl. C07K 14/47; A61K 38/00; A61P 7/02; C07K 14/445

(52) U.S. Cl. CPC C07K 14/4703; A61P 7/02; C07K 14/445; A61K 38/00; C07K 2319/33; C07K 2319/74

(57) ABSTRACT

A conjugate comprising a P. falciparum erythrocyte membrane protein 1 (PfEMP1) CIDR^{α1.4} domain fused to a therapeutic agent.

### FIELD OF THE INVENTION

The invention relates to conjugate for use in localising a drug target to the vascular endothelium. The invention can be used, for example, when restoring haemostasis in individuals with defective haemostasis.

### BACKGROUND TO THE INVENTION

Diseases arising from dysfunction of the vasculature, including cardiovascular disease, diabetes, obesity, cancer, and severe inflammatory diseases, such as COVID-19, arise from disruption of blood vessel health and integrity and in particular, their altered interaction with blood components.

Protein C is a blood enzyme that is activated in response to clot formation. Once converted to its active form (activated protein C; APC), it has an important role in regulating coagulation, where it acts as a ‘brake’ on clot formation. The rate of APC generation is dependent upon protein C interaction with its receptor, the endothelial cell protein C receptor (EPCR), which is expressed on the surface of blood vessels and positions protein C for optimal activation by another enzyme-receptor complex (thrombin-20) thrombomodulin complex). Importantly, however, APC must detach from EPCR to subsequently exert its anticoagulant activity, as binding to EPCR completely prevents APC anticoagulant activity due to the requirement for APC binding to cell surface phospholipids via its γ-carboxyglutamic acid domain, which is blocked by EPCR binding.

Normal functioning of the endothelial protein C receptor (EPCR) is therefore critical for normal haemostasis and control of vascular inflammation. Recently, the protein C pathway was also shown to be defective in Plasmodium falciparum-infected cerebral malaria patients. Moreover, EPCR was identified as a crucial binding site for vascular cytoadherence of P. falciparum-infected red blood cells.

Individuals with inherited bleeding disorders exhibit an increased tendency to bleed uncontrollably after injury. Individuals with severe haemophilia A exhibit an increased tendency to bleed uncontrollably after injury. In addition, they are at particular risk of spontaneous bleeding into joints, which in turn increases the likelihood of developing inflammatory joint disease.

Current therapies for individuals with inherited and acquired bleeding disorders generally involve the administration of procoagulant proteins/agents. Haemophilia A and B therapy entails replacement of the missing clotting factor with recombinant or plasma-derived purified factor VIII (FVIII)/factor IX (FIX). However, ˜30% of individuals receiving replacement therapy develop inhibitory antibodies against the replacement protein. Individuals with haemophilia who develop inhibitory antibodies are normally treated with haemostatic agents that ‘bypass’ the requirement for functional FVIII. Licensed ‘bypass agents’, also used for the treatment of individuals who do not have haemophilia but are at high risk of bleeding, include recombinant factor VIIa (FVIIa) (NovoSeven; Novo Nordisk, US2017296634; US2013137157) and FVIII Bypassing Agent (FEIBA; Shire). These agents are, however, expensive, must be administered frequently and exhibit significant inter-individual efficacy.

Recently, novel approaches to generate therapies to stimulate haemostasis have been described. For individuals with severe haemophilia, these include a bi-specific antibody that mimics the cofactor function of activated FVIII in the intrinsic tenase complex to facilitate factor Xa (FXa) generation (Hemlibra/Emicizumab/ACE910; Roche, Pat No. EP1688488) and a recombinant engineered FVIII-von Willebrand factor fragment chimera that stabilises FVIII and prolongs its half-life (BIVV001; Bioverativ/Biogen, Pat. No. MY159135). For the treatment of individuals with haemophilia with anti-FVIII inhibitors and individuals who do not have haemophilia but have an acquired bleeding risk, additional therapies have been developed. For example, an engineered recombinant factor Va (FVa) molecule that exhibits enhanced stability and is impervious to normal enzymatic regulation, a zymogen-like activated factor X (FX) not susceptible to normal protease inhibitors (FXa^{I16L}; Pfizer; Pat. No. US2009175931). In addition, other approaches to suppress endogenous anticoagulant activity to boost blood clotting are also being evaluated, including short inhibitory RNA directed against antithrombin (fitusuran; Alnylam, Pat. No. TW201726148) a monoclonal antibody directed against tissue factor pathway inhibitor (PF-06741086; Pfizer; Pat. No. TW201718659) and an 30 engineered serine protease inhibitor with increased specificity against APC enzymatic activity (KRK α₁AT, ApcinteX; Pat. No, MX2016007711). Notably, due to their mechanism of action, some of these therapeutic approaches have the potential to induce thrombosis in treated individuals. Consequently, although both inhibitor bypass agents and general haemostatic agents exist and are in clinical use, no single product dominates the market.

In addition, many individuals exhibit transient but potentially life-threatening bleeding in the event of childbirth, surgery, or trauma without inherited haemostatic defects. For example, major bleeding causes ˜40% of deaths associated with major trauma. Furthermore, postpartum haemorrhage (PPH) is the most common form of major obstetric haemorrhage and occurs in up to 18% of live births. PPH is almost certainly under-estimated due to difficulty in objective measurements of obstetric bleeding. To date, there are limited therapeutic interventions to restore haemostasis in these affected individuals. Recombinant factor VIIa (FVIIa) has shown some efficacy in attenuating bleeding refractory to standard treatment, but existing treatments to restore haemostasis in these settings are often ineffective, expensive, and used without strong evidence to support their use.

WO 2013/177705 describes P. falciparum VAR2CSA fusions with a therapeutic moiety (e.g. toxins). WO 2015/095952 describes VAR2CSA conjugated to a compound.

It is an object of the subject invention to overcome at least one of the above-mentioned problems.

### SUMMARY OF THE INVENTION

The Applicants have generated a novel conjugate that targets therapeutic agents or therapeutic targets directly to the vascular endothelium. The novel conjugate comprises either replacing the endothelial cell protein C receptor (EPCR)-binding GIa domain of the therapeutic agent or target with a CIDRα1.4 domain of a cytoadhesion protein expressed by the P. falciparum parasite, P. falciparum erythrocyte membrane protein 1 (PfEMP1), or linking the agent or target with the PfEMP1 CIDRα1.4 domain. The Applicants have shown that the recombinant fusion protein binds to EPCR on endothelial cells with >100-fold increased affinity compared to wild type. The novel recombinant fusion protein has no anticoagulant activity and instead promotes haemostasis

One example of the conjugate is a novel recombinant fusion protein that promotes haemostasis and binds with high affinity to EPCR to block adhesion of P. falciparum-infected red blood cell binding to endothelial cells. This synthetic enzyme consists of a truncated activated protein C (APC^{CIDR}), characterised by replacement of its endogenous EPCR-binding GIa domain with a CIDRα1.4 domain of a cytoadhesion protein expressed by the P. falciparum parasite, P. falciparum erythrocyte membrane protein 1 (PfEMP1).

It has been shown by the Applicant that APC^{CIDR} possessed no independent anticoagulant activity and did not block endogenously generated APC anticoagulant activity in normal pooled plasma. APC^{CIDR} potently impedes protein C activation on the surface of blood vessel endothelial cells, suggesting it could ‘re-balance’ haemostasis in individuals with high risk of bleeding. Notably, APC^{CIDR} still retains normal cytoprotective signalling activity, suggesting it could block P. falciparum-infected red blood cell binding to blood vessel endothelial cells, without compromising essential protein C pathway function.

Recently, a subset of a clonally variant cytoadhesion protein expressed by the P. falciparum parasite, P. falciparum erythrocyte membrane protein 1 (PfEMP1), was shown to bind EPCR with up to 200-fold higher affinity than that described for APC (K_{D}˜0.3 nM). PfEMP1 consists of a combination of Duffy binding-like and cysteine-rich interdomain region (CIDR) domains that are sub-classified based on sequence similarity. PfEMP1 variants containing CIDRα1.1 and 1.4-1.8 domains bind EPCR at a similar site to protein C/APC, but with much higher affinity. EPCR is used by the parasite-infected red blood cells to bind to brain blood vessels and promote onset of life-threatening cerebral malaria. Consequently, novel biologics that can inhibit P. falciparum-infected red blood cell binding to endothelial cells, without compromising the anti-inflammatory properties of the protein C pathway, may have utility as an adjunctive treatment for cerebral malaria.

Collectively, these characteristics indicate that APC^{CIDR} represents a novel haemostatic agent with the potential to restore clotting in people with uncontrolled bleeding. Furthermore, the ultra-high affinity for EPCR makes APC^{CIDR} a potential adjunctive therapy to minimise vascular dysfunction in individuals with diseases such as cerebral malaria and other inflammatory conditions.

Another example of the conjugate of the claimed invention is a factor VII(a) fusion molecule based on FVIIa, which was designed by replacing its endogenous EPCR-binding GIa domain with the CIDRα1.4 domain of PfEMP (FVIIa^{CIDRα1.4}) to facilitate endothelial cell adhesion. This novel fusion protein exhibits enhanced functional properties, including increased endothelial protein C receptor (EPCR) affinity, haemostatic properties, and enhanced anti-inflammatory signalling properties.

According to the present invention, there is provided an isolated recombinant fusion protein comprising a therapeutic agent fused to a PfEMP1 CIDR domain as defined by SEQ ID NO. 1, or functional variant thereof.

According to the present invention, there is provided a conjugate comprising a P. falciparum erythrocyte membrane protein 1 (PfEMP1) CIDR^{α1.4} domain fused to a therapeutic agent. In one aspect, the PfEMP1 CIDR^{α1.4} domain is defined by SEQ ID NO. 1, or a functional variant thereof. In one aspect, the therapeutic agent is selected from an enzyme, an antibody, a small molecule inhibitor, a protein, and a drug.

In one aspect, the conjugate is an isolated recombinant fusion protein comprising a truncated APC molecule (SEQ ID NO. 6) fused to a PfEMP1 CIDR domain (SEQ ID NO. 1).

In one aspect, the isolated recombinant fusion protein comprising the truncated APC molecule fused to the PfEMP1 CIDR domain has a high affinity for endothelial cell protein C receptor.

In one aspect, there is provided an isolated recombinant fusion protein comprising a truncated APC molecule (SEQ ID NO. 6) fused to a PfEMP1 CIDR domain (SEQ ID NO. 1) for use as a hemostatic agent.

In one aspect, there is provided an isolated recombinant fusion protein comprising a truncated APC molecule (SEQ ID NO. 6) fused to a PfEMP1 CIDR domain (SEQ ID NO. 1 for use to treat vascular dysfunction in subjects. In certain aspects, the subject has cerebral malaria.

In one aspect, the isolated recombinant fusion protein comprising the truncated APC molecule (SEQ ID NO. 6) fused to the PfEMP1 CIDR domain (SEQ ID NO. 1) is defined by SEQ ID NO. 7. The truncated activated protein C is characterised by replacement of its endogenous EPCR-binding GIa domain with a PfEMP1 CIDRα1.4 domain.

In one aspect, the isolated recombinant fusion protein comprising the truncated APC molecule fused to the PfEMP1 CIDR domain is defined by SEQ ID NO. 7, or functional variant thereof.

In one aspect, there is provided a nucleic acid encoding the recombinant fusion protein described above as defined by SEQ ID NO. 8, or a variant thereof encoding a functional variant.

In one aspect, there is provided an isolated recombinant fusion protein comprising a Factor VIIa molecule (SEQ ID NO. 9) fused to a PfEMP1 CIDR domain (SEQ ID NO. 1) for use as a hemostatic agent. In this instance, the Factor VIIa molecule (SEQ ID NO. 9) is a truncated Factor VIIa molecule (SEQ ID NO. 9).

In one aspect, there is provided an isolated recombinant fusion protein comprising a Factor VIIa molecule (SEQ ID NO. 9) fused to a PfEMP1 CIDR domain (SEQ ID NO. 1) for use to treat vascular dysfunction in subjects. In this instance, the Factor VIIa molecule (SEQ ID NO. 9) is a truncated Factor VIIa molecule (SEQ ID NO. 9). In certain aspects, the subject has cerebral malaria.

In one aspect, the isolated recombinant fusion protein comprising the Factor VIIa molecule (SEQ ID NO. 9) fused to the PfEMP1 CIDR domain (SEQ ID NO. 1) is defined by SEQ ID NO. 11. The Factor VIIa molecule is characterised by replacement of its endogenous EPCR-binding GIa domain with a PfEMP1 CIDRα1.4 domain. In this instance, the Factor VIIa molecule (SEQ ID NO. 9) is a truncated Factor VIIa molecule (SEQ ID NO. 9).

In one aspect, the isolated recombinant fusion protein comprising the Factor VIIa molecule fused to the PfEMP1 CIDR domain is defined by SEQ ID NO. 11, or functional variant thereof. In this instance, the Factor VIIa molecule (SEQ ID NO. 9) is a truncated Factor VIIa molecule (SEQ ID NO. 9).

In one aspect, there is provided a nucleic acid encoding the recombinant fusion protein as defined by SEQ ID NO. 11, is encoded by SEQ ID NO. 12, or a variant thereof encoding a functional variant.

In one aspect, there is provided an isolated recombinant fusion protein comprising a meizothrombin molecule (SEQ ID NO. 13) fused to a PfEMP1 CIDR domain (SEQ ID NO. 1) for use as a hemostatic agent.

In one aspect, there is provided an isolated recombinant fusion protein comprising a meizothrombin molecule (SEQ ID NO. 13) fused to a PfEMP1 CIDR domain (SEQ ID NO. 1) for use to treat vascular dysfunction in subjects. In one aspect, the subject could have cerebral malaria.

In one aspect, the isolated recombinant fusion protein comprising the meizothrombin molecule (SEQ ID NO. 13) fused to the PfEMP1 CIDR domain (SEQ ID NO. 1) is defined by SEQ ID NO. 15. The meizothrombin molecule is characterised by replacement of its endogenous EPCR-binding GIa domain with a PfEMP1 CIDRα1.4 domain.

In one aspect, the isolated recombinant fusion protein comprising the meizothrombin molecule fused to the PfEMP1 CIDR domain is defined by SEQ ID NO. 15, or functional variant thereof.

In one aspect, there is provided a nucleic acid encoding the recombinant fusion protein as defined by SEQ ID NO. 15, is encoded by SEQ ID NO. 16, or a variant thereof encoding a functional variant.

In one aspect there is provided an expression vector comprising the nucleic acid described above. Preferably, the expression vector is selected from the group consisting of an adenovirus-associated virus (AAV) vector, a retroviral vector, an adenoviral vector, a plasmid, or a lentiviral vector. Preferably, said AAV vector comprises an AAV1, AAV2, AAV3, AAV4, AAV5, AAV6, AAV7, AAV8, AAV9, AAV10, AAV11, RhIO, Rh74 or AAV-218 AAV serotype.

Preferably, the expression vector above further comprises an intron, an expression control element, one or more adeno-associated virus (AAV) inverted terminal repeats (ITRs) and/or a filler polynucleotide sequence. More preferably, the intron is within or flanks the nucleic acid encoding FVIII variant, or wherein the expression control element is operably linked to the nucleic acid encoding the isolated recombinant fusion protein described above, or wherein the AAV ITR(s) flanks the 5′ or 3′ terminus of the nucleic acid encoding the isolated recombinant fusion protein, or wherein the filler polynucleotide sequence flanks the 5′ or 3′ terminus of the nucleic acid the isolated recombinant fusion protein described above.

In one aspect, the expression control element comprises a constitutive or regulatable control element, or a tissue-specific expression control element or promoter. In one aspect, for example, the expression control element comprises an element that confers expression in the heart. In one aspect, for example, the expression control element comprises a myosin light chain-2v promoter or mutant myosin light chain-2v promoter In one aspect, for example, the expression control element comprises an element that confers expression in liver. In one aspect, for example, the expression control element comprises a TTR promoter or mutant TTR promoter.

In one aspect, the ITR comprises one or more ITRs of any of: AAV1, AAV2, AAV3, AAV4, AAV5, AAV6, AAV7, AAV8, AAV9, AAV10, AAVI I, RhIO, Rh74 or AAV-218 AAV serotypes, or a combination thereof.

In one aspect, there is provided a pharmaceutical composition comprising the isolated recombinant fusion protein described above and a biologically acceptable carrier.

In one aspect, the pharmaceutical composition above is suitable for administration to a subject.

In one aspect, there is provided an isolated recombinant fusion protein comprising the truncated APC molecule fused to the PfEMP1 CIDR domain as described above, or the nucleic acid described above encoding the recombinant fusion protein, for use in a method of treating a hemostatic disorder.

In one aspect, there is provided an isolated recombinant fusion protein comprising the Factor VIIa molecule fused to the PfEMP1 CIDR domain as described above, or the nucleic acid described above encoding the recombinant fusion protein, for use in a method of preventing a hemostatic disorder.

In one aspect, there is provided an isolated recombinant fusion protein comprising the meizothrombin molecule fused to the PfEMP1 CIDR domain as described above, or the nucleic acid described above encoding the recombinant fusion protein, for use in a method of preventing a hemostatic disorder.

In one aspect, the hemostatic disorder is selected from the group consisting of hemophilia A, hemophilia B, FVII deficiency, FV deficiency, FX deficiency, FXI deficiency, Glanzmann's thrombasthenia, Bernard-Soulier syndrome, von Willebrand diseases, hemophilic arthropathy, bleeding of unknown cause, menorrhagia, rare inherited platelet function disorders, bleeding associated with trauma, injury, thrombosis, thrombocytopenia, stroke, coagulopathy, disseminated intravascular coagulation (DIC) and over-anticoagulation treatment disorders.

In one aspect, there is provided an isolated recombinant fusion protein comprising the truncated APC molecule fused to the PfEMP1 CIDR domain as described above, or the nucleic acid described above encoding the recombinant fusion protein, for use in a method of treating or preventing vascular dysfunction in subjects.

In one aspect, there is provided an isolated recombinant fusion protein comprising the Factor VIIa molecule fused to the PfEMP1 CIDR domain as described above, or the nucleic acid described above encoding the recombinant fusion protein, for use in a method of treating or preventing vascular dysfunction in subjects.

In one aspect, there is provided an isolated recombinant fusion protein comprising the meizothrombin molecule fused to the PfEMP1 CIDR domain as described above, or the nucleic acid described above encoding the recombinant fusion protein, for use in a method of treating or preventing vascular dysfunction in subjects.

In one aspect, there is provided a formulation comprising the isolated recombinant fusion protein described above for use in the methods of treatment described above.

Vascular dysfunction occurs in many diseases or conditions, such as acute inflammatory diseases and thrombotic disorders. Examples of thrombotic disease include deep vein thrombosis (DVT), ischemic stroke, Paget-Schroetter disease, Budd-Chiara syndrome, portal vein thrombosis, renal vein thrombosis, cerebral venous sincus thrombosis, jugular vein thrombosis, cavernous sinus thrombosis, arterial thrombosis, myocardial infarction, limb ischemia, hepatic artery thrombosis, thrombotic thrombocytopenia purpura (TTP). Examples of acute inflammatory diseases are diabetes (including type 1 diabetes), cardiovascular disease (ischemic heart disease, cardiac hypertrophy; myocardial infarction; stroke; arteriosclerosis; and heart failure), arthritis (such as osteoarthritis, rheumatoid arthritis, fibromyalgia, gout, childhood arthritis, lupus), allergies, asthma, chronic obstructive pulmonary disease (COPD), psoriasis, acne, vasculitis, inflammatory bowel disease (such as colitis, ulcerative colitis, and Crohn's disease), multiple sclerosis, chronic inflammatory demyelinating polyneuropathy, Guillain-Barre syndrome, Grave's disease, myasthenia gravis, cerebral malaria, cancer, celiac disease, glomerulonephritis, hepatitis, cryopyrinopathies or cryopyrin-associated periodic syndromes (CAPS) (a group of three rare autoinflammatory diseases that includes familial cold autoinflammatory syndrome (FCAS), Muckle-Wells syndrome (MWS) and chronic infantile neurologic cutaneous articular syndrome (CINCA)), disease caused by rhinoviruses (for example, the common cold), and coronaviruses (for example, Severe acute respiratory syndrome coronavirus 1 (SARS-COV-1 or Severe acute respiratory syndrome (SARS)), Severe acute respiratory syndrome coronavirus 2 (SARS-COV-2 or COVID-19), and Middle East respiratory syndrome-related coronavirus (MERS-COV or MERS)).

### Definitions

As used herein, the term “dysregulated hemostasis” or “dysfunctional hemostasis” should be understood to mean where the ability of the subject to prevent excessive blood loss and induce thrombus formation is abnormal such that excessive bleeding or excessive thrombosis occurs.

As used herein, the term “subject” or “patient” or refers to any mammal. The patient is preferably a human but can also be a mammal in need of veterinary treatment, for example, a cat, a dog, a cow, a horse, a sheep, a goat, a donkey, a horse, a bull, a calf, a lamb, a foal, a kid, a monkey, an ape, a zebra, a giraffe, a lion, a tiger, a cheetah, a lemur, a gibbon, and other mammals commonly found in zoos and wildlife parks.

For administration to a subject, the agents can be provided in pharmaceutically acceptable compositions. These pharmaceutically acceptable compositions comprise a therapeutically effective amount of the agent, formulated together with one or more pharmaceutically acceptable carriers (additives) and/or diluents. The pharmaceutical compositions can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally (e.g. as a nasal spray or suppository); or (9) nasally. Additionally, agents can be implanted into a patient or injected using a drug delivery system. See, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Guidance for formulations can be found in e.g. Remington: The Science and Practice of Pharmacy by Alfonso R. Gelmaro (Ed.) 20th edition: Dec. 15, 2000, Lippincott, Williams $ Wilkins, ISBN: 0683306472.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The amount of agent which can be combined with a carrier material to produce a single dosage form will generally be that amount of the agent which produces a therapeutic effect. Generally, out of one hundred percent, this amount will range from about 0.1% to 99% of agent, preferably from about 5% to about 70%, most preferably from 10% to about 30%.

Formulations suitable for parenteral administration conveniently include sterile aqueous preparation of the active agent which is preferably isotonic with the blood of the recipient. Thus, such formulations may conveniently contain distilled water, 5% dextrose in distilled water or saline. Useful formulations also include concentrated solutions or solids containing the agent which upon dilution with an appropriate solvent give a solution suitable for parental administration above.

For enteral administration, an agent can be incorporated into an inert carrier in discrete units such as capsules, cachets, tablets or lozenges, each containing a predetermined amount of the active agent; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active agent in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active agent with any suitable carrier.

A syrup or suspension may be made by adding the active agent to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS (N, N-bis(3-D-gluconamidopropyl)-cholamide), chlorobutanol, octoxynol-9, benzyl alcohol, phenols, cresols, and alkyl alcohols. In one embodiment, the oral absorption enhancer may be sodium lauryl sulfate.

As used herein, the term “administer(s)” or “administering” refers to the placement of a composition into a subject by a method or route which results in at least partial localization of the composition at a desired site such that desired effect is produced. An agent or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In the specification, the term “inflammatory condition” should be understood to mean immune-related conditions resulting in allergic reactions, myopathies and abnormal inflammation and non-immune related conditions having causal origins in inflammatory processes. Examples include as sepsis, acne, autoimmune conditions, autoinflammatory condition, chronic prostatitis, diverticulitis, cancer, heart disease, cerebral malaria, and the like.

In the specification, the term “autoinflammatory condition” should be understood to mean a group of diseases characterised by seemingly unprovoked episodes of fever and inflammation of skin, joints, serosal surfaces and other organ involvement including the nervous system. Examples of autoinflammatory conditions include allergy, asthma, autoimmune conditions, celiac disease, glomerulonephritis, hepatitis, cryopyrinopathies or cryopyrin-associated periodic syndromes (CAPS) (a group of three rare autoinflammatory diseases that includes familial cold autoinflammatory syndrome (FCAS), Muckle-Wells syndrome (MWS) and chronic infantile neurologic cutaneous articular syndrome (CINCA)), and the like.

In the specification, the term “autoimmune condition” should be understood to mean a condition in which your immune system mistakenly attacks your body. Examples of autoimmune conditions include asthma, inflammatory bowel diseases, lupus, rheumatoid arthritis, multiple sclerosis, Type 1 diabetes, Chronic inflammatory demyelinating polyneuropathy, Guillain-Barre syndrome, psoriasis, vasculitis, Grave's disease, myasthenia gravis, Hashimoto's thyroiditis, and the like.

In the specification, the term “inflammatory bowel disease” should be understood to mean disorders that involve chronic inflammation of the digestive tract. Examples of inflammatory bowel disease include colitis, ulcerative colitis, and Crohn's disease.

In the specification, the term “cancer” should be understood to mean a cancer selected from the group comprising node-negative, ER-positive breast cancer; early stage, node positive breast cancer; multiple myeloma, prostate cancer, glioblastoma, lymphoma, fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumour; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumour; cervical cancer; uterine cancer; testicular tumour; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukaemias. Also included are metastases selected from the group comprising: bone metastases; lung metastases; liver metastases; bone marrow metastases; breast metastases; and brain metastases.

In the specification, the term “hemostatic agent” should be understood to mean an antihemorrhagic (antihemorrhagic) agent that promotes hemostasis (stops bleeding. Antihemorrhagic agents used in medicine have various mechanisms of action: Systemic drugs work by inhibiting fibrinolysis or promoting coagulation.

In the specification, the term “heart disease” should be understood to mean cardiovascular disease selected from the group comprising: ischemic heart disease, cardiac hypertrophy; myocardial infarction; stroke; arteriosclerosis; and heart failure.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

The terms “polypeptide,” “peptide” and “protein” refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acids.

As used herein, “variant polypeptides” may comprise conservatively substituted sequences, meaning that one or more amino acid residues is replaced by different residues, and that the conservatively substituted polypeptide retains a desired biological activity, that is essentially equivalent to that of the native polypeptide. Examples of conservative substitutions include substitution of amino acids that do not alter the secondary and/or tertiary structure of the polypeptide. Other examples involve substitution of amino acids that have not been evolutionarily conserved. One or more polypeptide sequences from non-human species can be aligned with, for example, human using methods well known to one of ordinary skill in the art to determine which residues are conserved and which tolerate more variability. Advantageously, in some embodiments, these conserved amino acids are not altered when generating conservatively substituted sequences.

Any given amino acid may be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. For example, APC^{CIDR} polypeptides comprising conservative amino acid substitutions can be tested in any one of the assays described herein to confirm that a desired haemostatic activity of a native APC is retained.

Amino acids may be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Conservative substitutions may include, for example: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in U.S. patent application No. 20070213292; and certain ribonucleoside that are is linked between the 2′-oxygen and the 4′-carbon atoms with a methylene unit as described in U.S. Pat. No. 6,268,490.

In the specification, the term “conjugate” should be understood to mean a compound formed by the joining of two or more chemical compounds. In this case, the conjugate can be a protein joined to a non-protein moiety (for example, a drug, small molecule inhibitor) or a protein joined to a protein moiety (for example, an enzyme (such as APC, meizothrombin, or FVIIa), an antibody, a small molecule inhibitor) or a protein joined to a protein or non-protein component of a drug formulation (for example, cylcodextrins, r vesicles, nanoparticles or solid-lipid liposomes, phospholipids, extracellular nanoparticles).

In the specification, the term “enzyme” should be understood to mean a protein that acts a biological catalyst that accelerate chemical reactions. Examples include activated protein C (APC), Factor VIIa, ADAM metallopeptidase with thrombospondin type 1 motif 13 (ADAMTS13), tissue plasminogen activator (t-PA), plasmin, and meizothrombin.

In the specification, the term “antibody” should be understood to mean a protein that is used by the immune system to identify and neutralize foreign objects such as pathogenic bacteria and viruses. Examples of antibodies (or fragments thereof) used herein include those targeting Intercellular adhesion molecule-1 (ICAM-1), vascular cell adhesion molecule-1 (VCAM-1), E-selectin, P-selectin, thrombomodulin, CD13, Angiotensin-Converting Enzyme 2 (ACE2), α-integrins (CD49a-f, ITGA7, ITGA8, ITGA9, ITGA10, ITGA11, CD11D, CD103, CD11a, CD11b, CD51, CD41 and CD11c) and β-integrins (CD29, CD18, CD61, CD104, ITGB5, ITGB6, ITGB7, and ITGB8).

In the specification, the term “small molecule inhibitor” should be understood to mean a drug that can enter cells easily because it has a low molecular weight. Once inside the cells, it can affect other molecules, such as proteins, and may cause cancer cells to die. Examples of small molecule inhibitors include, for example, Lenalidomide (Revlimid®-used to treat myeloma and blood disorders called myelodysplastic syndromes), apixaban (an anticoagulant), and desmopressin (used to treat diabetes insipidus, bedwetting, hemophilia A, von Willebrand disease, and high blood urea levels).

In the specification, the term “drug” should be understood to mean any chemical substance that causes a change in an organism's physiology or psychology when consumed. Examples include nucleic acid-based therapies such as RNAi, mRNA, siRNA, gene therapy, and gene editing constructs; cellular and tissue therapies such as chimeric antigen receptor (CAR) T cell therapy, or exosomes; chemotherapeutics; immunomodulators; anticoagulants, and the like.

In the specification, it should be understood that the therapeutic is not labelled with a his-tag or other tag.

The terms “decrease”, “reduced”, “reduction”, “decrease”, or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%. In one embodiment, there is a 100% decrease (e.g., absent level as compared to a reference sample).

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means two standard deviations (2SD) below normal, or lower, concentration of the marker. The term refers to statistical evidence that there is a difference. It is defined as the probability of deciding to reject the null hypothesis when the null hypothesis is actually true. The decision is often made using the p-value.

Sequences
(PfEMP1 CIDR^{α1.4} HB3var03_HB3 domain)
SEQ ID NO. 1
PDCGVECKNETCTPKTVIYPDCGKNEKYEPPGDAKNTEINVINSG
DKEGYIFEKLSEFCTNENNENGKNYEQWKCYYDNKKNNNKCKMEI
NIANSKLKNKITSFDEFFDFWVRKLLIDTIKWETELTYCINNTDV
TDCNKCNKNCVCFDKWVKQKEDEWTNIMKLFTNKHDIPKKYYLNI
NDLFDSFFFQVIYKFNEGEAKWNELKENLKKQIASSKANNGTKDS
EAAIKVLFNHIKEIATICKDNNTNEGC
(PfEMP1 CIDR^{α1.4} HB3var03_HB3 domain)
SEQ ID NO. 2
ccggattgcggcgtggaatgcaaaaacgaaacctgcaccccgaaa
accgtgatttatccggattgcggcaaaaacgaaaaatatgaaccg
ccgggcgatgcgaaaaacaccgaaattaacgtgattaacagcggc
gataaagaaggctatatttttgaaaaactgagcgaattttgcacc
aacgaaaacaacgaaaacggcaaaaactatgaacagtggaaatgc
tattatgataacaaaaaaaacaacaacaaatgcaaaatggaaatt
aacattgcgaacagcaaactgaaaaacaaaattaccagctttgat
gaattttttgatttttgggtgcgcaaactgctgattgataccatt
aaatgggaaaccgaactgacctattgcattaacaacaccgatgtg
accgattgcaacaaatgcaacaaaaactgcgtgtgctttgataaa
tgggtgaaacagaaagaagatgaatggaccaacattatgaaactg
tttaccaacaaacatgatattccgaaaaaatattatctgaacatt
aacgatctgtttgatagctttttttttcaggtgatttataaattt
aacgaaggcgaagcgaaatggaacgaactgaaagaaaacctgaaa
aaacagattgcgagcagcaaagcgaacaacggcaccaaagatagc
gaagcggcgattaaagtgctgtttaaccatattaaagaaattgcg
accatttgcaaagataacaacaccaacgaaggctgctaa
(human protein C)
SEQ ID NO. 3
atgtggcagctgaccagcctgctgctgtttgtggcgacctggggc
attagcggcaccccggcgccgctggatagcgtgtttagcagcagc
gaacgcgcgcatcaggtgctgcgcattcgcaaacgcgcgaacagc
tttctggaagaactgcgccatagcagcctggaacgcgaatgcatt
gaagaaatttgcgattttgaagaagcgaaagaaatttttcagaac
gtggatgataccctggcgttttggagcaaacatgtggatggcgat
cagtgcctggtgctgccgctggaacatccgtgcgcgagcctgtgc
tgcggccatggcacctgcattgatggcattggcagctttagctgc
gattgccgcagcggctgggaaggccgcttttgccagcgcgaagtg
agctttctgaactgcagcctggataacggcggctgcacccattat
tgcctggaagaagtgggctggcgccgctgcagctgcgcgccgggc
tataaactgggcgatgatctgctgcagtgccatccggcggtgaaa
tttccgtgcggccgcccgtggaaacgcatggaaaaaaaacgcagc
catctgaaacgcgataccgaagatcaggaagatcaggtggatccg
cgcctgattgatggcaaaatgacccgccgcggcgatagcccgtgg
caggtggtgctgctggatagcaaaaaaaaactggcgtgcggcgcg
gtgctgattcatccgagctgggtgctgaccgcggcgcattgcatg
gatgaaagcaaaaaactgctggtgcgcctgggcgaatatgatctg
cgccgctgggaaaaatgggaactggatctggatattaaagaagtg
tttgtgcatccgaactatagcaaaagcaccaccgataacgatatt
gcgctgctgcatctggcgcagccggcgaccctgagccagaccatt
gtgccgatttgcctgccggatagcggcctggcggaacgcgaactg
aaccaggcgggccaggaaaccctggtgaccggctggggctatcat
agcagccgcgaaaaagaagcgaaacgcaaccgcacctttgtgctg
aactttattaaaattccggtggtgccgcataacgaatgcagcgaa
gtgatgagcaacatggtgagcgaaaacatgctgtgcgcgggcatt
ctgggcgatcgccaggatgcgtgcgaaggcgatagcggcggcccg
atggtggcgagctttcatggcacctggtttctggtgggcctggtg
agctggggcgaaggctgcggcctgctgcataactatggcgtgtat
accaaagtgagccgctatctggattggattcatggccatattcgc
gataaagaagcgccgcagaaaagctgggcgccg
(human protein C)
SEQ ID NO. 4
MWQLTSLLLFVATWGISGTPAPLDSVFSSSERAHQVLRIRKRANS
FLEELRHSSLERECIEEICDFEEAKEIFQNVDDTLAFWSKHVDGD
QCLVLPLEHPCASLCCGHGTCIDGIGSFSCDCRSGWEGRFCQREV
SFLNCSLDNGGCTHYCLEEVGWRRCSCAPGYKLGDDLLQCHPAVK
FPCGRPWKRMEKKRSHLKRDTEDQEDQVDPRLIDGKMTRRGDSPW
QVVLLDSKKKLACGAVLIHPSWVLTAAHCMDESKKLLVRLGEYDL
RRWEKWELDLDIKEVFVHPNYSKSTTDNDIALLHLAQPATLSQTI
VPICLPDSGLAERELNQAGQETLVTGWGYHSSREKEAKRNRTFVL
NFIKIPVVPHNECSEVMSNMVSENMLCAGILGDRQDACEGDSGGP
MVASFHGTWFLVGLVSWGEGCGLLHNYGVYTKVSRYLDWIHGHIR
DKEAPQKSWAP
(truncated human protein C)
SEQ ID NO. 5
atgtggcagctgaccagcctgctgctgtttgtggcgacctggggc
attagcggcaccccggcgccgctggatagcgtgtttagcagcagc
gaacgcgcgcatcaggtgctgcgcattcgcaaacgccatccgtgc
gcgagcctgtgctgcggccatggcacctgcattgatggcattggc
agctttagctgcgattgccgcagcggctgggaaggccgcttttgc
cagcgcgaagtgagctttctgaactgcagcctggataacggcggc
tgcacccattattgcctggaagaagtgggctggcgccgctgcagc
tgcgcgccgggctataaactgggcgatgatctgctgcagtgccat
ccggcggtgaaatttccgtgcggccgcccgtggaaacgcatggaa
aaaaaacgcagccatctgaaacgcgataccgaagatcaggaagat
caggtggatccgcgcctgattgatggcaaaatgacccgccgcggc
gatagcccgtggcaggtggtgctgctggatagcaaaaaaaaactg
gcgtgcggcgcggtgctgattcatccgagctgggtgctgaccgcg
gcgcattgcatggatgaaagcaaaaaactgctggtgcgcctgggc
gaatatgatctgcgccgctgggaaaaatgggaactggatctggat
attaaagaagtgtttgtgcatccgaactatagcaaaagcaccacc
gataacgatattgcgctgctgcatctggcgcagccggcgaccctg
agccagaccattgtgccgatttgcctgccggatagcggcctggcg
gaacgcgaactgaaccaggcgggccaggaaaccctggtgaccggc
tggggctatcatagcagccgcgaaaaagaagcgaaacgcaaccgc
acctttgtgctgaactttattaaaattccggtggtgccgcataac
gaatgcagcgaagtgatgagcaacatggtgagcgaaaacatgctg
tgcgcgggcattctgggcgatcgccaggatgcgtgcgaaggcgat
agcggcggcccgatggtggcgagctttcatggcacctggtttctg
gtgggcctggtgagctggggcgaaggctgcggcctgctgcataac
tatggcgtgtataccaaagtgagccgctatctggattggattcat
ggccatattcgcgataaagaagcgccgcagaaaagctgggcgccg
(truncated human protein C)
SEQ ID NO. 6
MWQLTSLLLFVATWGISGTPAPLDSVESSSERAHQVLRIRKRHPC
ASLCCGHGTCIDGIGSFSCDCRSGWEGRFCQREVSFLNCSLDNGG
CTHYCLEEVGWRRCSCAPGYKLGDDLLQCHPAVKFPCGRPWKRME
KKRSHLKRDTEDQEDQVDPRLIDGKMTRRGDSPWQVVLLDSKKKL
ACGAVLIHPSWVLTAAHCMDESKKLLVRLGEYDLRRWEKWELDLD
IKEVFVHPNYSKSTTDNDIALLHLAQPATLSQTIVPICLPDSGLA
ERELNQAGQETLVTGWGYHSSREKEAKRNRTFVLNFIKIPVVPHN
ECSEVMSNMVSENMLCAGILGDRQDACEGDSGGPMVASFHGTWFL
VGLVSWGEGCGLLHNYGVYTKVSRYLDWIHGHIRDKEAPQKSWAP
(truncated human APC molecule fused to the
PfEMP1 CIDR domain)
SEQ ID NO. 7
MRLAVGALLVCAVLGLCLPDCGVECKNETCTPKTVIYPDCGKNEK
YEPPGDAKNTEINVINSGDKEGYIFEKLSEFCTNENNENGKNYEQ
WKCYYDNKKNNNKCKMEINIANSKLKNKITSFDEFFDFWVRKLLI
DTIKWETELTYCINNTDVTDCNKCNKNCVCFDKWVKQKEDEWTNI
MKLFTNKHDIPKKYYLNINDLFDSFFFQVIYKENEGEAKWNELKE
NLKKQIASSKANNGTKDSEAAIKVLFNHIKEIATICKDNNTNEGC
GDQCLVLPLEHPCASLCCGHGTCIDGIGSFSCDCRSGWEGRFCQR
EVSFLNCSLDNGGCTHYCLEEVGWRRCSCAPGYKLGDDLLQCHPA
VKFPCGRPWKRMEKKRSHLKRDTEDQEDQVDPRLIDGKMTRRGDS
PWQVVLLDSKKKLACGAVLIHPSWVLTAAHCMDESKKLLVRLGEY
DLRRWEKWELDLDIKEVFVHPNYSKSTTDNDIALLHLAQPATLSQ
TIVPICLPDSGLAERELNQAGQETLVTGWGYHSSREKEAKRNRTF
VLNFIKIPVVPHNECSEVMSNMVSENMLCAGILGDRQDACEGDSG
GPMVASFHGTWFLVGLVSWGEGCGLLHNYGVYTKVSRYLDWIHGH
IRDKEAPQKSWAP
(truncated human APC molecule fused to the
PfEMP1 CIDR domain)
SEQ ID NO. 8
atgcgcctggcggtgggcgcgctgctggtgtgcgcggtgctgggc
ctgtgcctgccggattgcggcgtggaatgcaaaaacgaaacctgc
accccgaaaaccgtgatttatccggattgcggcaaaaacgaaaaa
tatgaaccgccgggcgatgcgaaaaacaccgaaattaacgtgatt
aacagcggcgataaagaaggctatatttttgaaaaactgagcgaa
ttttgcaccaacgaaaacaacgaaaacggcaaaaactatgaacag
tggaaatgctattatgataacaaaaaaaacaacaacaaatgcaaa
atggaaattaacattgcgaacagcaaactgaaaaacaaaattacc
agctttgatgaattttttgatttttgggtgcgcaaactgctgatt
gataccattaaatgggaaaccgaactgacctattgcattaacaac
accgatgtgaccgattgcaacaaatgcaacaaaaactgcgtgtgc
tttgataaatgggtgaaacagaaagaagatgaatggaccaacatt
atgaaactgtttaccaacaaacatgatattccgaaaaaatattat
ctgaacattaacgatctgtttgatagctttttttttcaggtgatt
tataaatttaacgaaggcgaagcgaaatggaacgaactgaaagaa
aacctgaaaaaacagattgcgagcagcaaagcgaacaacggcacc
aaagatagcgaagcggcgattaaagtgctgtttaaccatattaaa
gaaattgcgaccatttgcaaagataacaacaccaacgaaggctgc
ggcgatcagtgcctggtgctgccgctggaacatccgtgcgcgagc
ctgtgctgcggccatggcacctgcattgatggcattggcagcttt
agctgcgattgccgcagcggctgggaaggccgcttttgccagcgc
gaagtgagctttctgaactgcagcctggataacggcggctgcacc
cattattgcctggaagaagtgggctggcgccgctgcagctgcgcg
ccgggctataaactgggcgatgatctgctgcagtgccatccggcg
gtgaaatttccgtgcggccgcccgtggaaacgcatggaaaaaaaa
cgcagccatctgaaacgcgataccgaagatcaggaagatcaggtg
gatccgcgcctgattgatggcaaaatgacccgccgcggcgatagc
ccgtggcaggtggtgctgctggatagcaaaaaaaaactggcgtgc
ggcgcggtgctgattcatccgagctgggtgctgaccgcggcgcat
tgcatggatgaaagcaaaaaactgctggtgcgcctgggcgaatat
gatctgcgccgctgggaaaaatgggaactggatctggatattaaa
gaagtgtttgtgcatccgaactatagcaaaagcaccaccgataac
gatattgcgctgctgcatctggcgcagccggcgaccctgagccag
accattgtgccgatttgcctgccggatagcggcctggcggaacgc
gaactgaaccaggcgggccaggaaaccctggtgaccggctggggc
tatcatagcagccgcgaaaaagaagcgaaacgcaaccgcaccttt
gtgctgaactttattaaaattccggtggtgccgcataacgaatgc
agcgaagtgatgagcaacatggtgagcgaaaacatgctgtgcgcg
ggcattctgggcgatcgccaggatgcgtgcgaaggcgatagcggc
ggcccgatggtggcgagctttcatggcacctggtttctggtgggc
ctggtgagctggggcgaaggctgcggcctgctgcataactatggc
gtgtataccaaagtgagccgctatctggattggattcatggccat
attcgcgataaagaagcgccgcagaaaagctgggcgccgtaa
(human Factor VIIa)
SEQ ID NO. 9
MVSQALRLLCLLLGLQGCLAAGGVAKASGGETRDMPWKPGPHRVF
VTQEEAHGVLHRRRRANAFLEELRPGSLERECKEEQCSFEEAREI
FKDAERTKLFWISYSDGDQCASSPCQNGGSCKDQLQSYICFCLPA
FEGRNCETHKDDQLICVNENGGCEQYCSDHTGTKRSCRCHEGYSL
LADGVSCTPTVEYPCGKIPILEKRNASKPQGRIVGGKVCPKGECP
WQVLLLVNGAQLCGGTLINTIWVVSAAHCFDKIKNWRNLIAVLGE
HDLSEHDGDEQSRRVAQVIIPSTYVPGTTNHDIALLRLHQPVVLT
DHVVPLCLPERTESERTLAFVRFSLVSGWGQLLDRGATALELMVL
NVPRLMTQDCLQQSRKVGDSPNITEYMFCAGYSDGSKDSCKGDSG
GPHATHYRGTWYLTGIVSWGQGCATVGHFGVYTRVSQYIEWLQKL
MRSEPRPGVLLRAPFP
(human Factor VIIa)
SEQ ID NO. 10
atggtgagccaggcgctgcgcctgctgtgcctgctgctgggcctg
cagggctgcctggcggcgggcggcgtggcgaaagcgagcggcggc
gaaacccgcgatatgccgtggaaaccgggcccgcatcgcgtgttt
gtgacccaggaagaagcgcatggcgtgctgcatcgccgccgccgc
gcgaacgcgtttctggaagaactgcgcccgggcagcctggaacgc
gaatgcaaagaagaacagtgcagctttgaagaagcgcgcgaaatt
tttaaagatgcggaacgcaccaaactgttttggattagctatagc
gatggcgatcagtgcgcgagcagcccgtgccagaacggcggcagc
tgcaaagatcagctgcagagctatatttgcttttgcctgccggcg
tttgaaggccgcaactgcgaaacccataaagatgatcagctgatt
tgcgtgaacgaaaacggcggctgcgaacagtattgcagcgatcat
accggcaccaaacgcagctgccgctgccatgaaggctatagcctg
ctggcggatggcgtgagctgcaccccgaccgtggaatatccgtgc
ggcaaaattccgattctggaaaaacgcaacgcgagcaaaccgcag
ggccgcattgtgggcggcaaagtgtgcccgaaaggcgaatgcccg
tggcaggtgctgctgctggtgaacggcgcgcagctgtgcggcggc
accctgattaacaccatttgggtggtgagcgcggcgcattgcttt
gataaaattaaaaactggcgcaacctgattgcggtgctgggcgaa
catgatctgagcgaacatgatggcgatgaacagagccgccgcgtg
gcgcaggtgattattccgagcacctatgtgccgggcaccaccaac
catgatattgcgctgctgcgcctgcatcagccggtggtgctgacc
gatcatgtggtgccgctgtgcctgccggaacgcacctttagcgaa
cgcaccctggcgtttgtgcgctttagcctggtgagcggctggggc
cagctgctggatcgcggcgcgaccgcgctggaactgatggtgctg
aacgtgccgcgcctgatgacccaggattgcctgcagcagagccgc
aaagtgggcgatagcccgaacattaccgaatatatgttttgcgcg
ggctatagcgatggcagcaaagatagctgcaaaggcgatagcggc
ggcccgcatgcgacccattatcgcggcacctggtatctgaccggc
attgtgagctggggccagggctgcgcgaccgtgggccattttggc
gtgtatacccgcgtgagccagtatattgaatggctgcagaaactg
atgcgcagcgaaccgcgcccgggcgtgctgctgcgcgcgccgttt
ccgtaa
(human Factor VIIa molecule fused to the
PfEMP1 CIDR domain)
SEQ ID NO. 11
MRLAVGALLVCAVLGLCLPDCGVECKNETCTPKTVIYPDCGKNEK
YEPPGDAKNTEINVINSGDKEGYIFEKLSEFCTNENNENGKNYEQ
WKCYYDNKKNNNKCKMEINIANSKLKNKITSFDEFFDEWVRKLLI
DTIKWETELTYCINNTDVTDCNKCNKNCVCFDKWVKQKEDEWTNI
MKLFTNKHDIPKKYYLNINDLFDSFFFQVIYKENEGEAKWNELKE
NLKKQIASSKANNGTKDSEAAIKVLFNHIKEIATICKDNNTNEGC
DGDQCASSPCQNGGSCKDQLQSYICFCLPAFEGRNCETHKDDQLI
CVNENGGCEQYCSDHTGTKRSCRCHEGYSLLADGVSCTPTVEYPC
GKIPILEKRNASKPQGRIVGGKVCPKGECPWQVLLLVNGAQLCGG
TLINTIWVVSAAHCFDKIKNWRNLIAVLGEHDLSEHDGDEQSRRV
AQVIIPSTYVPGTTNHDIALLRLHQPVVLTDHVVPLCLPERTESE
RTLAFVRESLVSGWGQLLDRGATALELMVLNVPRLMTQDCLQQSR
KVGDSPNITEYMFCAGYSDGSKDSCKGDSGGPHATHYRGTWYLTG
IVSWGQGCATVGHFGVYTRVSQYIEWLQKLMRSEPRPGVLLRAPF
P
(human Factor VIIa molecule fused to the
PfEMP1 CIDR domain)
SEQ ID NO. 12
atgcgcctggcggtgggcgcgctgctggtgtgcgcggtgctgggc
ctgtgcctgccggattgcggcgtggaatgcaaaaacgaaacctgc
accccgaaaaccgtgatttatccggattgcggcaaaaacgaaaaa
tatgaaccgccgggcgatgcgaaaaacaccgaaattaacgtgatt
aacagcggcgataaagaaggctatatttttgaaaaactgagcgaa
ttttgcaccaacgaaaacaacgaaaacggcaaaaactatgaacag
tggaaatgctattatgataacaaaaaaaacaacaacaaatgcaaa
atggaaattaacattgcgaacagcaaactgaaaaacaaaattacc
agctttgatgaattttttgatttttgggtgcgcaaactgctgatt
gataccattaaatgggaaaccgaactgacctattgcattaacaac
accgatgtgaccgattgcaacaaatgcaacaaaaactgcgtgtgc
tttgataaatgggtgaaacagaaagaagatgaatggaccaacatt
atgaaactgtttaccaacaaacatgatattccgaaaaaatattat
ctgaacattaacgatctgtttgatagctttttttttcaggtgatt
tataaatttaacgaaggcgaagcgaaatggaacgaactgaaagaa
aacctgaaaaaacagattgcgagcagcaaagcgaacaacggcacc
aaagatagcgaagcggcgattaaagtgctgtttaaccatattaaa
gaaattgcgaccatttgcaaagataacaacaccaacgaaggctgc
gatggcgatcagtgcgcgagcagcccgtgccagaacggcggcagc
tgcaaagatcagctgcagagctatatttgcttttgcctgccggcg
tttgaaggccgcaactgcgaaacccataaagatgatcagctgatt
tgcgtgaacgaaaacggcggctgcgaacagtattgcagcgatcat
accggcaccaaacgcagctgccgctgccatgaaggctatagcctg
ctggcggatggcgtgagctgcaccccgaccgtggaatatccgtgc
ggcaaaattccgattctggaaaaacgcaacgcgagcaaaccgcag
ggccgcattgtgggcggcaaagtgtgcccgaaaggcgaatgcccg
tggcaggtgctgctgctggtgaacggcgcgcagctgtgcggcggc
accctgattaacaccatttgggtggtgagcgcggcgcattgcttt
gataaaattaaaaactggcgcaacctgattgcggtgctgggcgaa
catgatctgagcgaacatgatggcgatgaacagagccgccgcgtg
gcgcaggtgattattccgagcacctatgtgccgggcaccaccaac
catgatattgcgctgctgcgcctgcatcagccggtggtgctgacc
gatcatgtggtgccgctgtgcctgccggaacgcacctttagcgaa
cgcaccctggcgtttgtgcgctttagcctggtgagcggctggggc
cagctgctggatcgcggcgcgaccgcgctggaactgatggtgctg
aacgtgccgcgcctgatgacccaggattgcctgcagcagagccgc
aaagtgggcgatagcccgaacattaccgaatatatgttttgcgcg
ggctatagcgatggcagcaaagatagctgcaaaggcgatagcggc
ggcccgcatgcgacccattatcgcggcacctggtatctgaccggc
attgtgagctggggccagggctgcgcgaccgtgggccattttggc
gtgtatacccgcgtgagccagtatattgaatggctgcagaaactg
atgcgcagcgaaccgcgcccgggcgtgctgctgcgcgcgccgttt
ccgtaa
(human meizothrombin molecule)
SEQ ID NO. 13
MAHVRGLQLPGCLALAALCSLVHSQHVFLAPQQARSLLQRVRRAN
TFLEEVRKGNLERECVEETCSYEEAFEALESSTATDVFWAKYTAC
ETARTPRDKLAACLEGNCAEGLGTNYRGHVNITRSGIECQLWRSR
YPHKPEINSTTHPGADLQENFCRNPDSSTTGPWCYTTDPTVRRQE
CSIPVCGQDQVTVAMTPQSEGSSVNLSPPLEQCVPDRGQQYQGRL
AVTTHGLPCLAWASAQAKALSKHQDENSAVQLVENFCRNPDGDEE
GVWCYVAGKPGDFGYCDLNYCEEAVEEETGDGLDEDSDRAIEGQT
ATSEYQTFFNPQTFGSGEADCGLRPLFEKKSLEDKTERELLESYI
DGRIVEGSDAEIGMSPWQVMLFRKSPQELLCGASLISDRWVLTAA
HCLLYPPWDKNFTENDLLVRIGKHSRTRYERNIEKISMLEKIYIH
PRYNWRENLDRDIALMKLKKPVAFSDYIHPVCLPDRETAASLLQA
GYKGRVTGWGNLKETWTANVGKGQPSVLQVVNLPIVERPVCKDST
RIRITDNMFCAGYKPDEGKRGDACEGDSGGPFVMKSPFNNRWYQM
GIVSWGEGCDRDGKYGFYTHVERLKKWIQKVIDQFGE
(human meizothrombin molecule)
SEQ ID NO. 14
atggcgcatgtgcgcggcctgcagctgccgggctgcctggcgctg
gcggcgctgtgcagcctggtgcatagccagcatgtgtttctggcg
ccgcagcaggcgcgcagcctgctgcagcgcgtgcgccgcgcgaac
acctttctggaagaagtgcgcaaaggcaacctggaacgcgaatgc
gtggaagaaacctgcagctatgaagaagcgtttgaagcgctggaa
agcagcaccgcgaccgatgtgttttgggcgaaatataccgcgtgc
gaaaccgcgcgcaccccgcgcgataaactggcggcgtgcctggaa
ggcaactgcgcggaaggcctgggcaccaactatcgcggccatgtg
aacattacccgcagcggcattgaatgccagctgtggcgcagccgc
tatccgcataaaccggaaattaacagcaccacccatccgggcgcg
gatctgcaggaaaacttttgccgcaacccggatagcagcaccacc
ggcccgtggtgctataccaccgatccgaccgtgcgccgccaggaa
tgcagcattccggtgtgcggccaggatcaggtgaccgtggcgatg
accccgcagagcgaaggcagcagcgtgaacctgagcccgccgctg
gaacagtgcgtgccggatcgcggccagcagtatcagggccgcctg
gcggtgaccacccatggcctgccgtgcctggcgtgggcgagcgcg
caggcgaaagcgctgagcaaacatcaggattttaacagcgcggtg
cagctggtggaaaacttttgccgcaacccggatggcgatgaagaa
ggcgtgtggtgctatgtggcgggcaaaccgggcgattttggctat
tgcgatctgaactattgcgaagaagcggtggaagaagaaaccggc
gatggcctggatgaagatagcgatcgcgcgattgaaggccagacc
gcgaccagcgaatatcagaccttttttaacccgcagacctttggc
agcggcgaagcggattgcggcctgcgcccgctgtttgaaaaaaaa
agcctggaagataaaaccgaacgcgaactgctggaaagctatatt
gatggccgcattgtggaaggcagcgatgcggaaattggcatgagc
ccgtggcaggtgatgctgtttcgcaaaagcccgcaggaactgctg
tgcggcgcgagcctgattagcgatcgctgggtgctgaccgcggcg
cattgcctgctgtatccgccgtgggataaaaactttaccgaaaac
gatctgctggtgcgcattggcaaacatagccgcacccgctatgaa
cgcaacattgaaaaaattagcatgctggaaaaaatttatattcat
ccgcgctataactggcgcgaaaacctggatcgcgatattgcgctg
atgaaactgaaaaaaccggtggcgtttagcgattatattcatccg
gtgtgcctgccggatcgcgaaaccgcggcgagcctgctgcaggcg
ggctataaaggccgcgtgaccggctggggcaacctgaaagaaacc
tggaccgcgaacgtgggcaaaggccagccgagcgtgctgcaggtg
gtgaacctgccgattgtggaacgcccggtgtgcaaagatagcacc
cgcattcgcattaccgataacatgttttgcgcgggctataaaccg
gatgaaggcaaacgcggcgatgcgtgcgaaggcgatagcggcggc
ccgtttgtgatgaaaagcccgtttaacaaccgctggtatcagatg
ggcattgtgagctggggcgaaggctgcgatcgcgatggcaaatat
ggcttttatacccatgtgtttcgcctgaaaaaatggattcagaaa
gtgattgatcagtttggcgaataa
(human meizothrombin molecule fused to the
PfEMP1 CIDR domain)
SEQ ID NO. 15
MRLAVGALLVCAVLGLCLHHHHHHPDCGVECKNETCTPKTVIYPD
CGKNEKYEPPGDAKNTEINVINSGDKEGYIFEKLSEFCTNENNEN
GKNYEQWKCYYDNKKNNNKCKMEINIANSKLKNKITSFDEFFDFW
VRKLLIDTIKWETELTYCINNTDVTDCNKCNKNCVCFDKWVKQKE
DEWTNIMKLFTNKHDIPKKYYLNINDLFDSFFFQVIYKENEGEAK
WNELKENLKKQIASSKANNGTKDSEAAIKVLFNHIKEIATICKDN
NTNEGCTACETARTPRDKLAACLEGNCAEGLGTNYRGHVNITRSG
IECQLWRSRYPHKPEINSTTHPGADLQENFCRNPDSSTTGPWCYT
TDPTVRRQECSIPVCGQDQVTVAMTPQSEGSSVNLSPPLEQCVPD
RGQQYQGRLAVTTHGLPCLAWASAQAKALSKHQDENSAVQLVENF
CRNPDGDEEGVWCYVAGKPGDFGYCDLNYCEEAVEEETGDGLDED
SDRAIEGQTATSEYQTFFNPQTFGSGEADCGLRPLFEKKSLEDKT
ERELLESYIDGRIVEGSDAEIGMSPWQVMLFRKSPQELLCGASLI
SDRWVLTAAHCLLYPPWDKNFTENDLLVRIGKHSRTRYERNIEKI
SMLEKIYIHPRYNWRENLDRDIALMKLKKPVAFSDYIHPVCLPDR
ETAASLLQAGYKGRVTGWGNLKETWTANVGKGQPSVLQVVNLPIV
ERPVCKDSTRIRITDNMFCAGYKPDEGKRGDACEGDSGGPFVMKS
PENNRWYQMGIVSWGEGCDRDGKYGFYTHVFRLKKWIQKVIDQFG
E
(human meizothrombin molecule fused to the
PfEMP1 CIDR domain)
SEQ ID NO. 16
atgcgcctggcggtgggcgcgctgctggtgtgcgcggtgctgggc
ctgtgcctgcatcatcatcatcatcatccggattgcggcgtggaa
tgcaaaaacgaaacctgcaccccgaaaaccgtgatttatccggat
tgcggcaaaaacgaaaaatatgaaccgccgggcgatgcgaaaaac
accgaaattaacgtgattaacagcggcgataaagaaggctatatt
tttgaaaaactgagcgaattttgcaccaacgaaaacaacgaaaac
ggcaaaaactatgaacagtggaaatgctattatgataacaaaaaa
aacaacaacaaatgcaaaatggaaattaacattgcgaacagcaaa
ctgaaaaacaaaattaccagctttgatgaattttttgatttttgg
gtgcgcaaactgctgattgataccattaaatgggaaaccgaactg
acctattgcattaacaacaccgatgtgaccgattgcaacaaatgc
aacaaaaactgcgtgtgctttgataaatgggtgaaacagaaagaa
gatgaatggaccaacattatgaaactgtttaccaacaaacatgat
attccgaaaaaatattatctgaacattaacgatctgtttgatagc
tttttttttcaggtgatttataaatttaacgaaggcgaagcgaaa
tggaacgaactgaaagaaaacctgaaaaaacagattgcgagcagc
aaagcgaacaacggcaccaaagatagcgaagcggcgattaaagtg
ctgtttaaccatattaaagaaattgcgaccatttgcaaagataac
aacaccaacgaaggctgcaccgcgtgcgaaaccgcgcgcaccccg
cgcgataaactggcggcgtgcctggaaggcaactgcgcggaaggc
ctgggcaccaactatcgcggccatgtgaacattacccgcagcggc
attgaatgccagctgtggcgcagccgctatccgcataaaccggaa
attaacagcaccacccatccgggcgcggatctgcaggaaaacttt
tgccgcaacccggatagcagcaccaccggcccgtggtgctatacc
accgatccgaccgtgcgccgccaggaatgcagcattccggtgtgc
ggccaggatcaggtgaccgtggcgatgaccccgcagagcgaaggc
agcagcgtgaacctgagcccgccgctggaacagtgcgtgccggat
cgcggccagcagtatcagggccgcctggcggtgaccacccatggc
ctgccgtgcctggcgtgggcgagcgcgcaggcgaaagcgctgagc
aaacatcaggattttaacagcgcggtgcagctggtggaaaacttt
tgccgcaacccggatggcgatgaagaaggcgtgtggtgctatgtg
gcgggcaaaccgggcgattttggctattgcgatctgaactattgc
gaagaagcggtggaagaagaaaccggcgatggcctggatgaagat
agcgatcgcgcgattgaaggccagaccgcgaccagcgaatatcag
accttttttaacccgcagacctttggcagcggcgaagcggattgc
ggcctgcgcccgctgtttgaaaaaaaaagcctggaagataaaacc
gaacgcgaactgctggaaagctatattgatggccgcattgtggaa
ggcagcgatgcggaaattggcatgagcccgtggcaggtgatgctg
tttcgcaaaagcccgcaggaactgctgtgcggcgcgagcctgatt
agcgatcgctgggtgctgaccgcggcgcattgcctgctgtatccg
ccgtgggataaaaactttaccgaaaacgatctgctggtgcgcatt
ggcaaacatagccgcacccgctatgaacgcaacattgaaaaaatt
agcatgctggaaaaaatttatattcatccgcgctataactggcgc
gaaaacctggatcgcgatattgcgctgatgaaactgaaaaaaccg
gtggcgtttagcgattatattcatccggtgtgcctgccggatcgc
gaaaccgcggcgagcctgctgcaggcgggctataaaggccgcgtg
accggctggggcaacctgaaagaaacctggaccgcgaacgtgggc
aaaggccagccgagcgtgctgcaggtggtgaacctgccgattgtg
gaacgcccggtgtgcaaagatagcacccgcattcgcattaccgat
aacatgttttgcgcgggctataaaccggatgaaggcaaacgcggc
gatgcgtgcgaaggcgatagcggcggcccgtttgtgatgaaaagc
ccgtttaacaaccgctggtatcagatgggcattgtgagctggggc
gaaggctgcgatcgcgatggcaaatatggcttttatacccatgtg
tttcgcctgaaaaaatggattcagaaagtgattgatcagtttggc
gaataa

### BRIEF DESCRIPTION OF THE DRAWINGS

The invention will be more clearly understood from the following description of an embodiment thereof, given by way of example only, with reference to the accompanying drawings, in which:—

FIG. 1: Design and characterisation of APC^{CIDRα1.4}: (A) A protein C fusion molecule (PC^{CIDRα1.4}) was designed which incorporated a cysteine-rich interdomain region (CIDRα) domain from the Plasmodium falciparum adhesion protein (PfEMP1) expressed by P. falciparum-infected erythrocytes to facilitate endothelial cell adhesion. This novel fusion protein was predicted to exhibit enhanced functional properties, including increased endothelial protein C receptor (EPCR) affinity and inhibitory activity against endogenous anticoagulant APC generation. (B) illustrates the novel construct that was created, consisting of an N-terminal transferrin propeptide fused to the P. falciparum HB3var03 clonal variant CIDRα1.4 and the human protein C coding region, minus the GIa domain region (amino acids 1-45). This PC^{CIDRα1.4} construct was cloned into a pcDNA3.4 plasmid before expression from HEK 293 cells and purification using size-exclusion chromatography. (C) illustrates a Western blot analysis of recombinant wild type PC and PC^{CIDRα1.4} performed on expressed material. Untreated and PNGase F-treated PC (lane 1 and 2) and PC^{CIDRα1.4} (lane 3 and 4) was detected using an anti-human PC monoclonal antibody, highlighting normal expression and N-linked glycosylation of both proteins.

FIG. 2: Unique functional properties of APC^{CIDRα1.4} include enhanced EPCR binding on endothelial cells. (A) illustrates the binding of fluorescently-labelled APC and APC^{CIDRα1.4} (both 1-50 nM) to human umbilical vein endothelial cells (HUVECs), assessed by flow cytometry; (B) illustrates the number of live cell binding events; and (C) illustrates the corresponding mean fluorescence intensity, which shows significantly enhanced APC^{CIDRα1.4} binding to endothelial cells, compared to that observed for wild type APC. (D) illustrates the analysis of APC^{CIDRα1.4} binding to endothelial cells at lower concentrations (lowest concentration tested, 0.1 nM) and revealed the presence of bound protease despite the presence of extremely low APC APC^{CIDRα1.4} concentrations.

FIG. 3: APC^{CIDRα1.4} has no anticoagulant activity and restricts APC generation. In contrast to wild type APC (a), incubation of APC^{CIDRα1.4} (b) had no anticoagulant effect on thrombin generation in normal platelet-poor plasma. This was further reflected in the impaired reduction in (c) endogenous thrombin potential and (d) peak thrombin by APC^{CIDRα1.4} compared to wild type APC. (e) Wild type protein C (PC) or PC^{CIDRα1.4} (100 nM) were co-incubated with the thrombin-soluble thrombomodulin (TM) complex (2 nM and 25 nM, respectively) for 30 mins, before hirudin was added to stop the reaction. Subsequently, co-incubation with an APC specific chromogenic substrate CS-21 (66) (1.25 mg/mL) was used to measure newly generated APC. PC^{CIDRα1.4} was found to be activated by thrombin in the presence of soluble thrombomodulin at the same rate as wild type PC. (f) Alternatively, human endothelial (EA.hy926) cells were treated with PC or PC^{CIDRα1.4} (100 nM) and thrombin (5 nM) for 30 mins, before hirudin was added and the supernatant co-incubated with CS-21 (66) (1.25 mg/mL) to measure APC generation. Detection of APC^{CIDRα1.4} generated by thrombin on the surface of endothelial cells that express thrombomodulin was minimal, largely due to the high affinity of generated APC^{CIDRα1.4} for cell surface EPCR prevented detection of APC^{CIDRα1.4} in cell supernatant. (g) To assess the impact of APC^{CIDRα1.4} on protein C generation, endothelial cells were preincubated with active-site blocked wild type APC, (PAPCWT; 1-200 nM) or with active-site blocked ^{P}APC^{CIDRα1.4} (1-200 nM) for 30 mins in TBS supplemented with 3 mM CaCl₂), 0.6 mM MgCl₂ and 1% (w/v) BSA, before being incubated with protein C (100 nM) and thrombin (FIIa, 5 nM) to initiate protein C activation for a further 30 mins. Hirudin was added to stop the reaction before the supernatant was removed, and the APC generated was measured using CS-21 (66) (1.25 mg/mL). Experiments were performed in triplicate and data are presented as mean±S.D. [*=p<0.05, ****=p<0.0001].

FIG. 4: β-arrestin recruitment to PAR1 activated by APC and APC^{CIDRα1.4} (a) HEK 293T cells expressing EPCR (PAR1-Trio^{EPCR+} cells) were transfected with 2 plasmids expressing (i) PAR1 fused to an individual green fluorescent protein (GFP) β strand (PAR1^{WT/β-11}), (ii) β-arrestin fused to a different individual GFP β strand (β-arrestin^{GFPDβ-10}) and (iii) GFP deficient in β strands^{GFPDβ-1-9} (HEK 293 TB). mCherry was co-expressed as a transfection marker. (b) HEK 293T6 cells were treated with APC and analysed for their ability to recruit β-arrestin 2 to the phosphorylated C-terminal tail of PAR1. GFP formation was measured by flow cytometry as a measure of activated PAR1-β-arrestin complex assembly. (c) Generation of mCherry⁺/GFP⁺ cells was dependent on PAR1 proteolysis as confirmed using inhibitors of thrombin-induced PAR1 proteolysis (hirudin), APC active site inhibitor (PPACK) and PAR1 mutants which block thrombin and APC cleavage sites. β-arrestin 2 recruitment to PAR1 activated by either wild type APC or APC^{CIDRα1.4} was assessed using this assay system and revealed >2-fold increased β-arrestin 2 recruitment to PAR1 activated by APC^{CIDRα1.4} compared to wild type APC.

FIG. 5: APC^{CIDRα1.4} mediates PAR1 proteolysis and endothelial cytoprotective signalling that is at least equivalent to that observed for wild type APC. HEK 293T cells were co-transfected with EPCR and (A) alkaline phosphatase (AP)-PAR1^{R41Q} or (B) AP-PAR1^{R46Q} constructs and treated with thrombin (FIIa, 10 nM), wild type APC (100 nM) or APC^{CIDRα1.4} (100 nM) for 3 hr before the AP activity in the cell supernatant was assessed using QUANTI blue AP substrate. Experiments were performed in triplicate and data are presented as mean±S.D. [**=p<0.01]. (C) Confluent human umbilical vein endothelial cells (HUVECs) on polycarbonate membrane permeable trans-well inserts were pre-treated with APC (5 nM) with 3 mM CaCl₂) and 0.6 mM MgCl₂ prior to treatment with thrombin (5 nM) for 10 mins. Endothelial cell barrier permeability was assessed by migration of Evans Blue (250 μL, 0.67 μg/mL) through the HUVEC layer into the outer chamber as measured by OD at 650 nm. Endothelial barrier permeability is presented as a percentage of maximum permeability induced by thrombin (i.e., 100% permeability). Experiments were performed in triplicate and data are presented as mean±S.D. [**=p<0.001]. (D) Using a similar approach to assess APC^{CIDRα1.4}-mediated EPCR occupancy-dependent protection against thrombin-induced endothelial cell barrier permeability, HUVECs were pre-treated with active-site blocked APC (5 nM), with 3 mM CaCl₂) and 0.6 mM MgCl₂ for 1 hr prior to treatment with thrombin (5 nM) for 3 hr. Endothelial cell barrier integrity was assessed using 250 μL of Evans Blue (0.67 μg/mL) as migration of the dye through the cell monolayer into the outer chamber, was measured at OD 650 nm to determine endothelial cell barrier permeability. Endothelial barrier permeability is presented as a percentage of maximum permeability induced by thrombin (i.e., 100% permeability). Experiments were performed in triplicate and data are presented as mean±S.D. [*=p<0.05].

FIG. 6: Design and characterisation of Factor VIIa^{CIDRα1.4}: (a) A fusion molecule based on Factor VIIa (FVIIa^{CIDRα1.4}) was designed which incorporated a cysteine-rich interdomain region (CIDRα) domain from the Plasmodium falciparum adhesion protein (PfEMP1) expressed by P. falciparum-infected erythrocytes to facilitate endothelial cell adhesion. This novel fusion protein was predicted to exhibit enhanced functional properties, including increased endothelial protein C receptor (EPCR) affinity, haemostatic properties, and enhanced anti-inflammatory signalling properties. (b) illustrates a Western blot analysis of recombinant FVIIa^{CIDRα1.4} performed on expressed material from stably transfected HEK 293 cells.

FIG. 7: Design and characterisation of Meizothrombin^{CIDRα1.4}: (a) A meizothrombin fusion molecule (mFIIa^{CIDRα1.4}) was designed which incorporated a cysteine-rich interdomain region (CIDRα) domain from the Plasmodium falciparum adhesion protein (PfEMP1) expressed by P. falciparum-infected erythrocytes to facilitate endothelial cell adhesion. This novel fusion protein was predicted to exhibit enhanced functional properties, including increased endothelial protein C receptor (EPCR) affinity and enhanced anti-inflammatory signalling properties. (b) illustrates the binding of fluorescently-labelled human meizothrombin (mFIIa) and mFIIa^{CIDRα1.4} (both 1-50 nM) to human umbilical vein endothelial cells (HUVECs), assessed by flow cytometry.

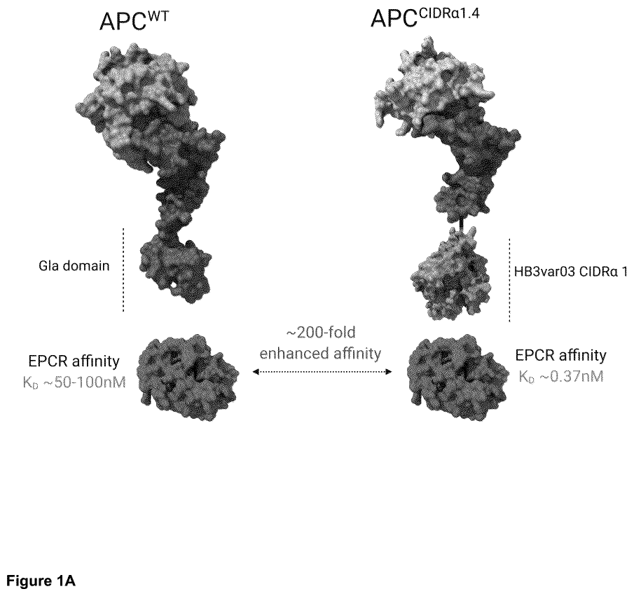

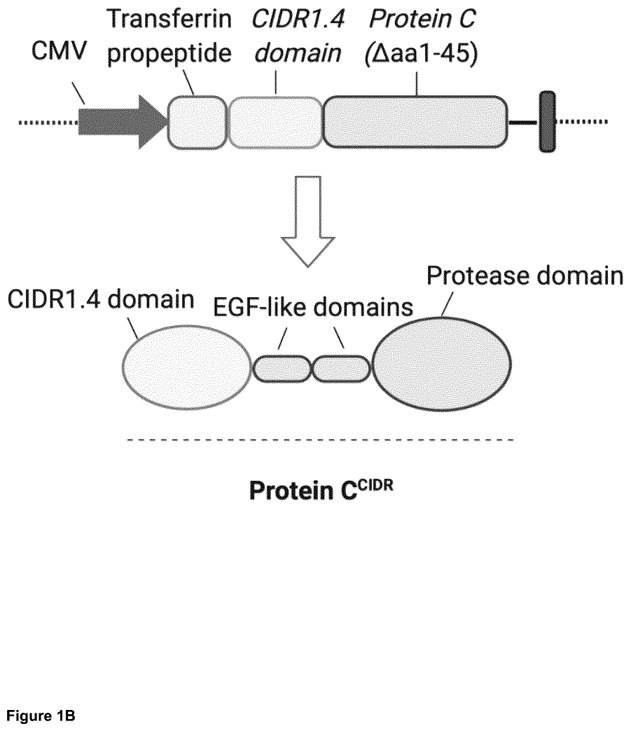

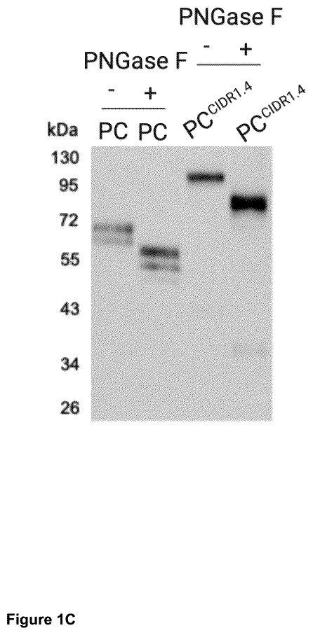

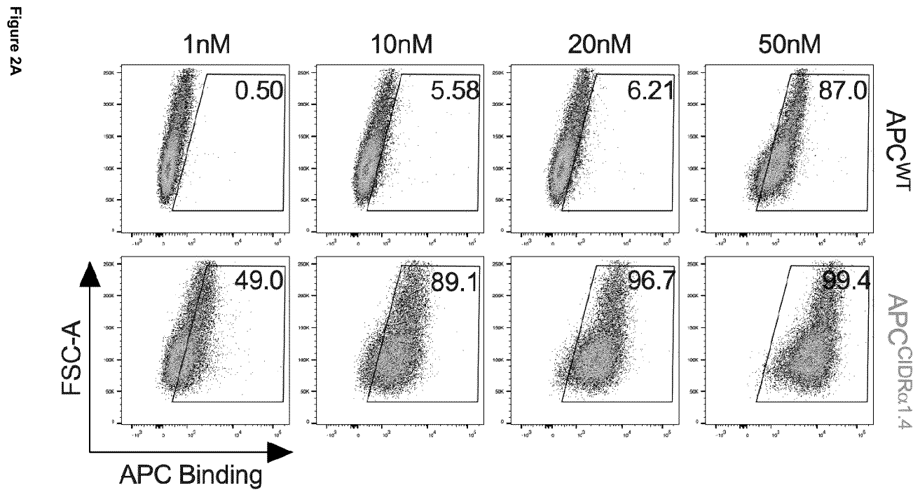

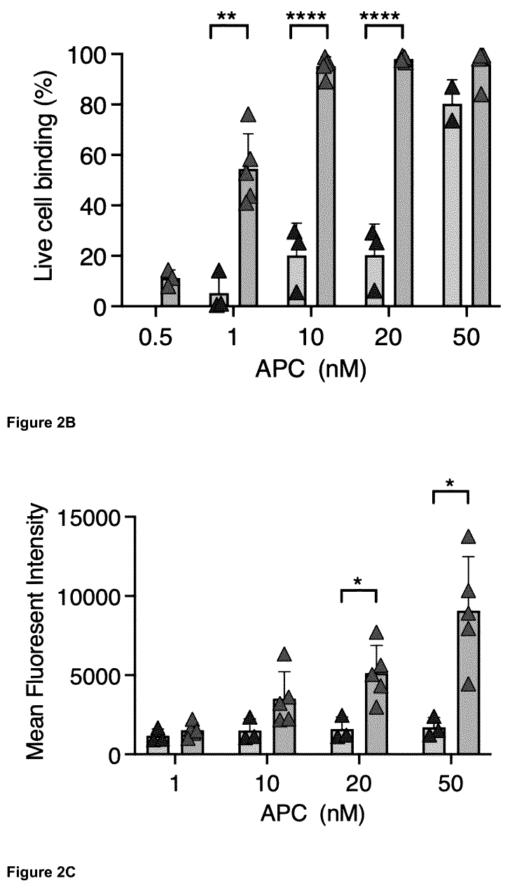

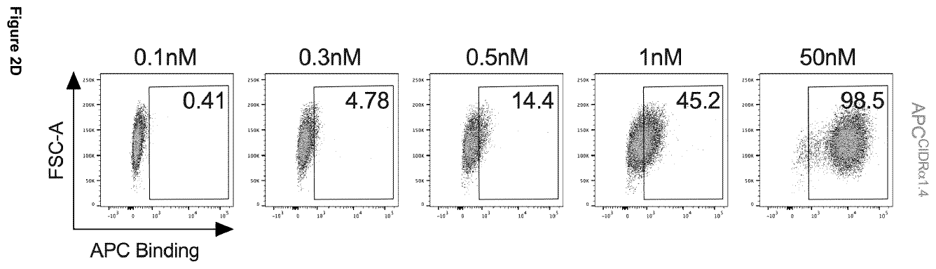

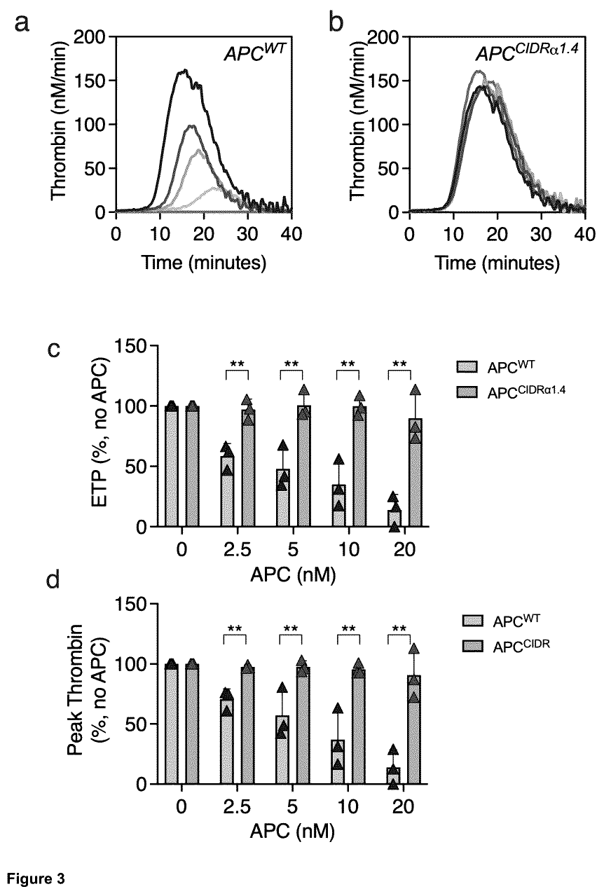

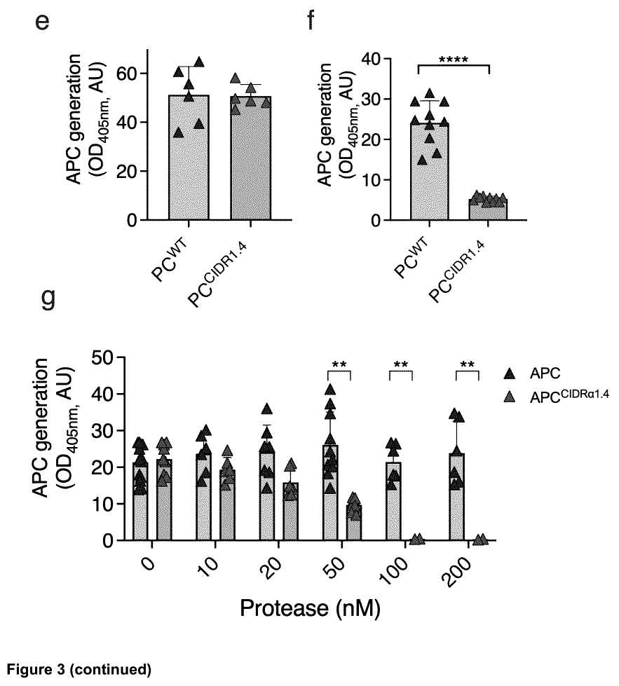

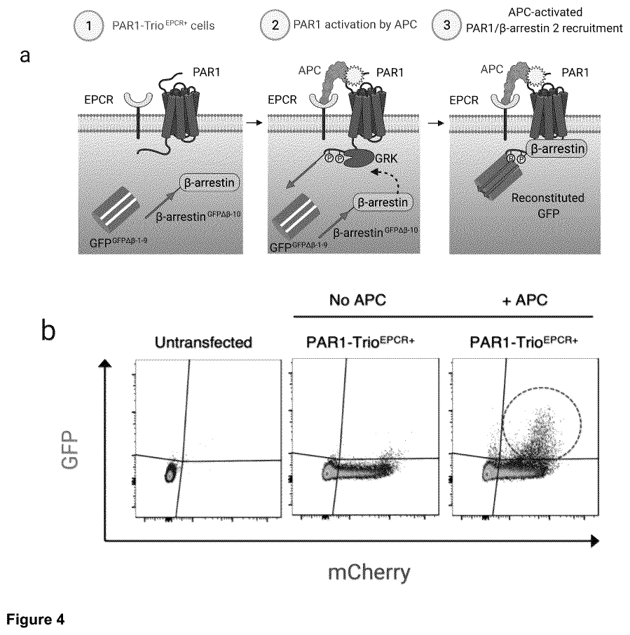

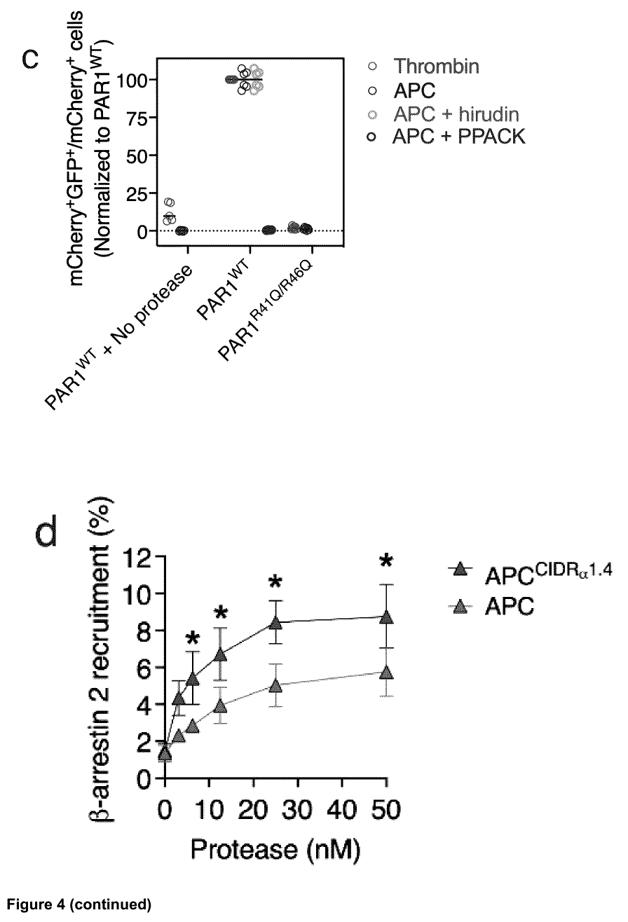

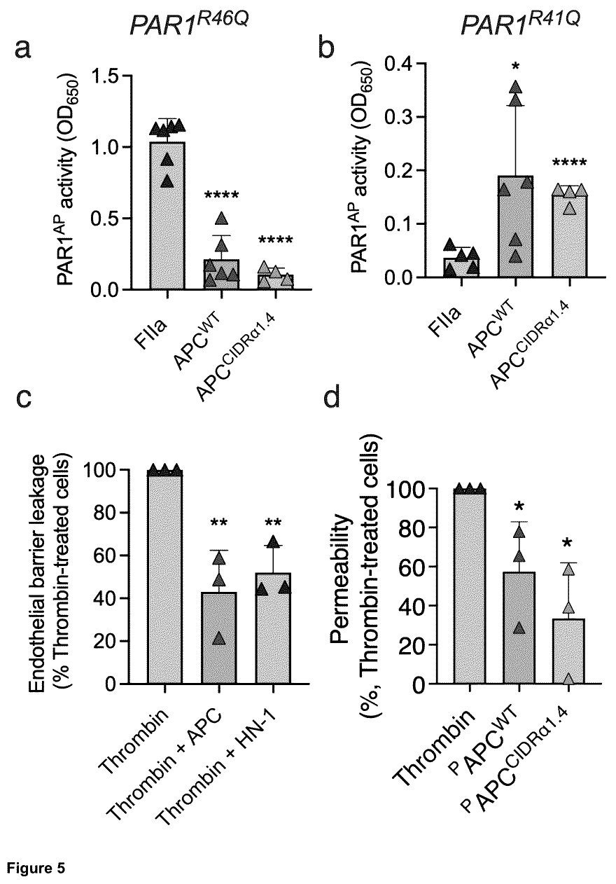

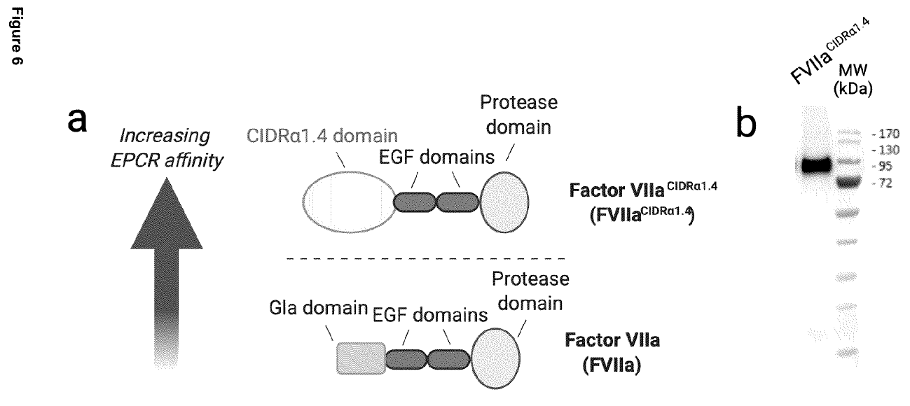

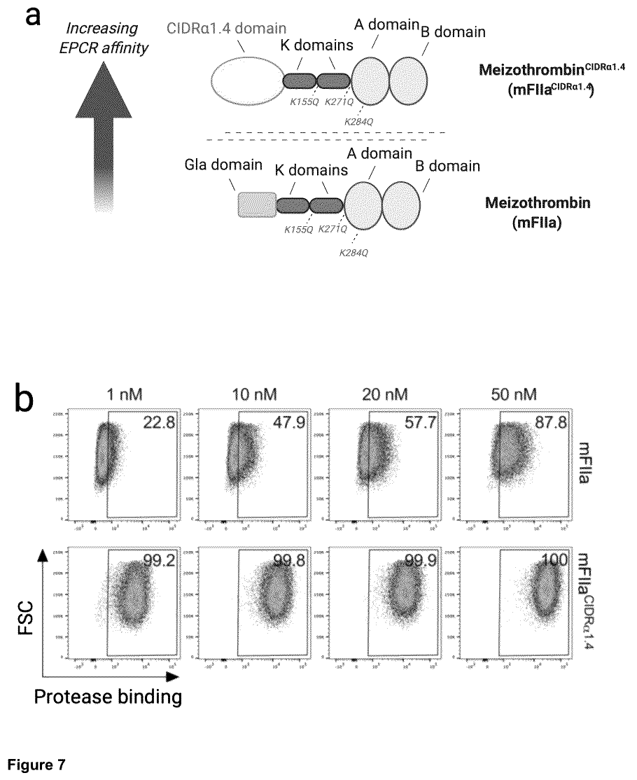

### DETAILED DESCRIPTION OF THE DRAWINGS

### Materials and Methods

### Recombinant Protein C^{CIDR} Expression

Human variants (PC and PC^{CIDR}) were generated using pcDNA3.1 (+) or pcDNA3.4 (+) template vectors. Stable transfection of HEK 293 cells was used for large-scale expression of each recombinant protein variant preparations. Geneticin sulphate (G418), antibiotic-selected colonies expressing each protein variant were expanded and the growth media containing recombinant protein was collected and purified using pseudo affinity and/or gel filtration chromatography.

### Expression and Concentration of Recombinant Protein C^{CIDR}

Successfully transfected protein-expressing colonies were expanded and stored by cryopreservation. Large-scale production of recombinant protein was achieved by expansion of transfected HEK 293 cells to full confluence in a multi surface cell culture vessel (500 mL HYPERflask; Corning). Growth media was replaced with 500 mL reduced serum media (opti-MEM), containing vitamin K (10 mg/mL) depending on the recombinant protein, and the cells were incubated for 3-5 days in a 37° C./5% CO₂ humidified incubator. The media was decanted and concentrated to ˜30 mL using a Pellicon XL tangential flow filtration (TFF, Millipore) system.

PC^{CIDR} containing concentrated media was passed over a HiLoad Superdex 16/600 75 pg column or a HiLoad Superdex 26/600 75 pg column packed with Superdex 75 prep grade resin. The HiLoad Superdex (16/600 or 26/600) 75 pg column was equilibrated with running buffer (50 mM Tris/150 mM NaCl, pH 7.5). Using a 1 mL super loop, the protein samples were injected and passed across the column. Eluted protein was collected into 2 mL fractions, pooled together and spin concentrated to ˜500 μL using an Amicon Ultra 15 ML (10 MWCO) spin concentrator (Merck Millipore). Quantification of protein concentration was obtained by human PC ELISA.

### Activation of PC^{CIDR}

PC^{CIDR} (1 μM) was activated using 3.58 U of biotinylated thrombin (2 μL, Novagen) in Ca²⁺-containing buffer (200 nM Tris/1.5 M NaCl/30 mM CaCl2, pH 7.5) and left rotating in a 37° C. incubator for 16 hr. The biotinylated thrombin was subsequently removed using streptavidin HP Spin Columns (GE Healthcare) that had been washed and equilibrated 3 times with the same Ca²⁺-containing buffer. Each APC preparation was incubated in a separate spin column for slow end-over-end mixing at room temperature for 60 mins. Activated enzymes were eluted by centrifugation at 1,000×g for 2 mins. When appropriate, hirudin (1U, Sigma) was added to APC preparations to inactivate any trace thrombin still remaining.

### Assessment of APC^{CIDR} Amidolytic Activity

The proteolytic activity of APC^{CIDR} was determined by their ability to hydrolyse an APC-specific chromogenic substrate, CS-21 (66) (1.25 mg/mL, Biophen) at an OD of 405 nm over 10 mins, taking readings every 30 sec using a spectrophotometer (VersaMax microplate reader, Molecular Devices). Each variant was serially diluted and measured against a standard of known APC (Haematologic Technologies).

### Western Blotting of Isolated APC^{CIDR}

100-500 ng of protein was diluted in NuPAGE loading buffer (Invitrogen, Life Technologies) with or without Reducing Agent (Invitrogen, Life Technologies). Samples were incubated at 70° C. for 10 mins before being resolved by SDS-PAGE using precast 10% polyacrylamide BisTris gels (Invitrogen, Life Technologies) for 40 mins at 200V. Using the Pierce Power Blotter semi-dry transfer system (Thermo Scientific), each sample was electrophoretically transferred onto a nitrocellulose membrane (Amersham Protran 0.45 NC, GE Healthcare). In order to block nonspecific binding sites, the membrane was incubated in 10 mL TBS with 5% (v/v) dried milk (Marvel) for 1 hr. The membrane was subsequently washed 3 times, 5 mins per wash, in TBS-0.1% Tween 20 (TBS-T) before being incubated overnight at 4° C. with a mouse anti-protein C primary antibody (Haematalogic Technologies Inc) diluted in 5% (v/v) dried milk (Marvel) in TBS. After 3 washes with TBS-T, the membrane was incubated with an appropriate HRP-conjugated secondary antibody (Haematalogic Technologies Inc) diluted in 5% milk for 1 hr at room temperature followed by a further 3 washes with TBS-T. The protein signals were enhanced by chemiluminescence (Pierce enhanced chemiluminescence (ECL) western blotting substrate, ThermoScientific) and detected using a chemiluminescence imager (Amersham imager 600, GE Healthcare).

### FACS Analysis of Coagulation Protease Binding to Endothelial Cell EPCR

To measure cell surface binding of coagulation proteases to the endothelial surface, APC and APC^{CIDR} were active-site blocked using biotinylated PPACK (P-APC, P-APC^{CIDR}). Co-incubation of allophycocyanin-conjugated streptavidin (Bio Sciences) resulted in fluorescently labelled proteins that were used to detect endothelial cell binding of each protease by FACS analysis. EA.hy926 cells were seeded at 5×10⁵/mL in a 12-well microtiter plate (CellStar) and left for 24 hr at 37° C. in a 5% CO₂ humidified incubator. Cells were removed from each well using a cell detachment buffer (PBS/5 mM EDTA) and incubated at 37° C. for 5 mins before being transferred into individual polypropylene FACS tubes for each condition, including controls (unstained cells, individual protein variant single stains and a live/dead single stain). To prevent non-specific binding of fluorescent IgG antibodies to cell surface Fc receptors, EA.hy926 cells were incubated with human Fc block (1:100 dilution, BD Biosciences). In addition, the monoclonal antibody RCR-252 (25 μg/ml, BD Biosciences) was used to prevent EPCR binding where described. Cells were centrifuged at 1500 rpm for 5 mins and incubated with active site-blocked APC species and HRP-conjugated streptavidin (Bio Sciences). All samples were kept in darkness and incubated at 37° C. for 30 mins. The cells were then washed with 1 mL of FACS buffer (PBS/2% FCS/3 mM CaCl₂)/0.6 mM MgCl₂) and centrifuged before being incubated with a LIVE/DEAD Fixable Green Stain for 488 nm excitation (1:100 dilution, ThermoFisher) for 25-30 mins to identify only live cells. These cells were then washed with FACS buffer, centrifuged and finally re-suspended in 300 μL of FACS buffer. Cells were then analysed using a FACSCanto II (BD Biosciences) flow cytometer and the data analysed using FlowJo software.

### Inhibition of PC Activation by APC^{CIDR} on the Surface of Endothelial Cells

The ability of PC variants to be activated by thrombin on endothelial cells was measured using the immortalised endothelial cell line, EA.hy926 cells (ATCC). EA.hy926 cells are derived from a hybrid clone between a HUVEC and thioguanine resistant A549 cells, a cell line derived from the adenocarcinome human alveolar basal epithelium. This assay was also modified to detect the ability of PC to be activated by thrombin after EPCR had been occupied by APC and APC^{CIDR} that had been active site-blocked with biotinylated D-Phe-Pro-Arg-chloromethylketone (PPACK) (P-APC and P-APC^{CIDR}). Cells were seeded into a 96-well microtiter plate (Cellstar) at a density of 2×10⁵/mL and grown to confluence over 48 hr. The cells were either washed twice with PBS and incubated with PC (100 nM) in TBS supplemented with 3 mM CaCl₂), 0.6 mM MgCl₂ and 1% (w/v) BSA or pre-treated with active-site blocked APC variants (P-APC/P-APC^{CIDR} (1-400 nM)) or the EPCR monoclonal antibody (RCR-252 (1-25 μg/mL)) for 30 mins before being incubated with PC, as described. 5 nM thrombin (Haematologic Technologies) was added to each well and incubated at 37° C. for 30 mins to initiate activation. The reaction was stopped by the addition of 1U hirudin (Sigma). Newly generated APC was assessed by determining APC amidolytic activity in the cell supernatant. 50 μL of the supernatant was added to 50 μL of the APC-specific chromogenic substrate CS01 (66) (1.25 mg/mL, Biophen). The rate of absorbance change was measured at 405 nm using a spectrophotometer (VersaMax microplate reader, Molecular Devices) and the kinetic parameters determined using Prism software.

### Assessment of APC Anticoagulant Activity in Protein C-Deficient Plasma

APC^{CIDR} anticoagulant function was assessed in protein C-deficient plasma using a Fluoroskan Ascent plate reader (Thermo lab Systems) in combination with Thrombinoscope software (Thrombinoscope). Briefly, 80 μL of protein C-deficient plasma (Enzyme Research Laboratories) was incubated with 20 μL of 5 μM platelet-poor plasma reagent (Thrombinoscope) containing soluble TF (5 μM) and phospholipids (4 μM) in the presence of wild type or APC^{CIDR} (2.5-20 nM). Thrombin generation was initiated by simultaneous addition of a fluorogenic thrombin substrate (Z-Gly-Gly-Arg-AMC-HCl, Thrombinoscope) and 100 mM CaCl₂) into each well. Thrombin generation was determined using a thrombin calibration standard. Measurements were taken at 20 sec intervals for 40 mins at 390 nm (excitation) and 460 nm (emission) wavelengths.

### Assessment of PAR1 Proteolysis by APC^{CIDR}

Assessment of PAR1 proteolysis was carried out using a recombinant PAR1 construct in which an alkaline phosphatase (AP) tag was fused N-terminal to predicted PAR1 cleavage sites (AP-PAR1). This construct was cloned into a pcDNA3.1 (+) plasmid. Proteolysis of this construct by APC resulted in liberation of the AP tag into the cell supernatant, which was then quantified using a colorimetric AP substrate. Two variants of the AP-PAR1 cDNA construct, synthesized by Genscript Biotech, were used to identify the specific site at which APC cleaved PAR1. Glu mutagenesis of the thrombin (Arg41) and APC (Arg46) PAR1 cleavage sites produced AP-PAR1 variants that were only cleaved at either the Arg41 or Arg46 cleavage sites (AP-PAR1^{R41Q}/AP-PAR1^{R46Q}). The experiments were carried out on HEK293T cells co-transfected with the mammalian expression vector pCMV6-AC expressing human EPCR.

HEK 293T cells were seeded into a 24-well microtiter plate (Cellstar) at a density of 2.5×10⁵ cells/mL. The cells were allowed to grow in a 37° C./5% CO₂ humidified incubator until they had reached 70-80% confluence (approximately 24 hr later). PAR1 variants containing an AP reporter (AP-PAR1/AP-PAR1^{R41Q}/AP-PAR1^{R46Q}) and EPCR plasmids were prepared for transfection by diluting 1 μg of plasmid cDNA in 100 μL of opti-MEM media (Gibco) along with the transfection reagent TurboFect (2 μL, Fisher Scientific). The mixture was vortexed and left to incubate at room temperature for 20-30 mins. Each confluent HEK 293T well was washed with 500 μL of sterile PBS before being left to incubate with the plasmid/TurboFect mixture for 6 hr. After this, the cells were washed again, and normal growth media was re-applied to allow the cells to reach full confluence. Culture medium was removed from each transfected well before being washed with sterile PBS. Serum-free MEM (Invitrogen) supplemented with 3 mM CaCl₂) and 0.6 mM MgCl₂ was used to incubate the cells with APC and APC^{CIDR} for 3 hr. AP activity was then measured by removing the supernatant and adding it to QUANTIBlue detection medium (Invivogen). The rate of AP substrate cleavage was measured using a spectrophotometer (VersaMax microplate reader, Molecular Devices) at 650 nm.

### FACS Analysis of β-Arrestin Recruitment by PAR1 Activated by APC

To better understand the effects of EPCR occupancy and downstream PAR1 signalling by different proteases, a tripartite fluorogenic assay was developed to assess recruitment of β-arrestin 1 or 2 to the C-terminal tail of PAR1 in HEK 293T cells. Assessment of β-arrestin recruitment following PAR1 activation was carried out using a PAR1 construct in which the green fluorescent protein (GFP) 11th β-strand was fused to the C-terminal end of PAR1 contained within the mammalian expression vector, pcDNA3.1 (+). This construct also co-expressed the 10th β-strand of GFP fused to either β-arrestin 1 or 2 along with the red fluorescent protein, mCherry, as a transfection marker. A T2A polyprotein cleavage sequence inserted between each protein coding sequence to 25 ensure production of three separate recombinant proteins. These experiments were carried out on HEK293T cells co-transfected with a second plasmid construct containing the remaining GFP β-sheets (1-9) in the mammalian expression vector, pcDNA3.1 (+) or with a third construct containing human EPCR in the pCMV6-AC plasmid. PAR1 activation brings the β-arrestin into close proximity and maturation of the GFP chromophore for GFP development. GFP maturation was used for facile analysis of these protein-protein interactions by flow cytometry.

HEK 293T cells were seeded into a 24-well microtiter plate (Cellstar) at a density of 5×10⁵ cells/mL and grown in a 37° C./5% CO₂ humidified incubator until they had reached 70-80% confluence (approximately 24 hr later). Once confluent, cells were transiently transfected by diluting 0.4 μg of each plasmid cDNA in 50 μL of opti-MEM media (Gibco) along with the transfection reagent TurboFect (2 μL, Fisher Scientific) for each well. Transiently transfected cells were treated with APC and APC^{CIDR} at different concentrations (10 pM-50 nM) in serum-free MEM (Invitrogen) supplemented with 3 mM CaCl₂) and 0.6 mM MgCl₂ for 2 hr, 24 hr after transfection, and assessment of GFP maturation was carried out by flow cytometry.

FACS analysis was achieved by detaching treated cells from each well using a cell detachment buffer (PBS/5 mM EDTA) and incubation at 37° C. for 5 mins. These cells were transferred into individual polypropylene FACS tubes for each condition, including an ‘untransfected’ and ‘no protease’ control, before being incubated with a LIVE/DEAD Fixable Near-IR Dead Stain for 635 nm excitation (1:100 dilution, ThermoFisher) for 15 mins. All samples were washed with 1 mL FACS buffer 2 (PBS, 5% FCS), centrifuged at 1,500 rpm for 5 mins and finally resuspended in 300 μL of FACS buffer. Cells were then analysed using a FACSCanto II (BD Biosciences) flow cytometer and the data was analysed using FlowJo software.

### APC^{CIDR}-Mediated Endothelial Barrier Protection Against Thrombin-Induced Leakage

In order to assess the barrier protective capacity of the recombinant enzymes generated, a previously described assay of endothelial cell barrier integrity was utilised. HUVECs were trypsinised and plated at a density of 2×10⁵ cells/mL on polycarbonate membrane transwell inserts (3 μM pore size, 12-mm diameter, Costar) contained within a 12-well plate. Plates were incubated at 37° C. in a 5% CO₂ incubator until full confluence was achieved (approximately 48 hr). The media was replaced in both chambers and left for a further 24 hr. The transwell inserts were drained and the cells were treated with serum-free endothelial growth media 2 (PromoCell), supplemented with 3 mM CaCl₂) and 0.6 mM MgCl₂, with APC or APC^{CIDR} for 3 hr. The cells were then treated with 5 nM thrombin for 10 mins to induce endothelial barrier permeability. The transwell inserts were drained and the cells were washed with sterile PBS before being incubated with Evans Blue (0.67 μg/mL, SigmaAldrich) in endothelial cell media with 0.4% BSA. Endothelial barrier permeability was determined by assessment of the increase in migration of Evans Blue dye into the outer chamber beneath the transwell insert at an OD of 650 nm, using a spectrophotometer (VersaMax microplate reader, Molecular Devices). Endothelial cell barrier permeability relative to thrombin-only treated cells was determined using the following equation (Equation 1):

$$Permeability ( \% ) = ( ( X - N ) / ( P - N ) ) \times 100 \qquad ( 1 )$$

Where X was the test sample, N was the endothelial media-treated negative control sample and P was the thrombin-treated positive control sample.

### Active Site-Blocked APC-Mediated Endothelial Barrier Protection by Thrombin

Occupancy of EPCR can recruit PAR1 to a barrier protective signalling pathway irrespective of the activating protease. Consequently, the endothelial cell barrier permeability assay was modified in order to assess the barrier protective capacity of thrombin when EPCR was occupied by the previously described novel variants. Briefly, HUVECs were grown to confluence on polycarbonate membrane transwell inserts and prepared for the assay. Cells were incubated with P-APC or P-APC^{CIDR} for 1 hr, after which 5 nM thrombin was added to each well and incubated for a further 3 hr to induce endothelial permeability. Endothelial barrier permeability was determined using the migration of Evans Blue as previously described.

### Statistical Analysis

All statistical tests were performed on the mean results of at least three independent experiments. Statistical analysis of experimental data was preformed using 2-tailed Student's t-test to determine if differences between samples were significant. Levels of significance are indicated using stars: *p≤0.05, **p≤0.01, ***p≤0.001, ****p≤0.0001.

### DISCUSSION

PfEMP1 CIDRα1.4 from the P. falciparum HB3var03 strain was shown to have the fastest association and slowest dissociation rate for EPCR binding compared to other CIDRα1 domains, resulting in sub nano-molar affinity, prompting its selection as a fusion partner with APC. APC and APC^{CIDR} binding to EPCR on the surface of endothelial cells was assessed. Endothelial cell binding was still clearly visible when <0.3 nM of APC^{CIDR} was used, whereas no APC binding was observed at <1 nM. These results demonstrate that APC^{CIDR} is therefore the first APC variant with enhanced affinity for EPCR and furthermore that CIDRα1.4 domain fusion to (A) PC does not noticeably impair CIDRα1.4 domain binding affinity for EPCR.

APC^{CIDR} displayed minimal plasma anticoagulant activity in an assay of tissue factor-dependent thrombin generation. The APC GIa domain is critical for APC anticoagulant function and mediates binding to both negatively charged phospholipids on the surface of activated cells proximal to vessel injury, protein S, both of which are necessary for FVa and FVIIIa proteolysis by APC to suppress thrombin generation.

The data presented herein also demonstrates that APC^{CIDR} cleaves PAR1 with comparable activity as APC and that absence of the APC GIa domain does not impede PAR1 proteolysis or modify the PAR1 proteolysis location if APC is localised via an alternative binding domain. Recent studies indicate that EPCR occupation by APC drives PAR1-dependent β-arrestin 2 recruitment and subsequent cytoprotective signalling outputs. This has been further underscored by data showing thrombin-mediated PAR1 cleavage causes β-arrestin 2 recruitment when EPCR is occupied by APC. Interestingly, although there was limited evidence that APC^{CIDR} mediated enhanced PAR1 proteolysis compared to APC, prolonged engagement of APC^{CIDR} with EPCR coupled with normal PAR1 proteolysis mediated ˜3-fold enhanced recruitment of β-arrestin 2 to APC^{CIDR}-activated PAR1 in HEK 293T β-arrestin 2 reporter cells. Collectively, these data indicate that EPCR binding regulates the nature of PAR1 signalling in endothelial cells when occupied by (A) PC.

APC^{CIDR} was found to mediate cytoprotective signalling activity in endothelial cells, as evidenced by protection of the endothelium from thrombin-induced endothelial cell barrier disruption. These data confirm findings by the Applicant relating to PAR1 proteolysis and β-arrestin recruitment induced by β-arrestin 2, that suggests that loss of the APC GIa domain and replacement with a distinct EPCR binding module does not ablate APC cytoprotective signalling activity.

EPCR occupancy by (A) PC can recruit thrombin-activated PAR1 to a barrier protective signalling pathway. Co-incubation of thrombin with proteolytically inactive APC and APC^{CIDR} resulted in significant reversal of thrombin-induced endothelial barrier disruption.

The unique properties of APC^{CIDR} may have potential application as an adjunctive therapy for the treatment of cerebral malaria. Cerebral malaria is a life-threating complication of P. falciparum infection, characterised by sequestration of infected erythrocytes to the endothelial cell surface of the brain microvasculature to cause inflammation and endothelial cell activation. Cytoadhesion of P. falciparum-infected erythrocytes to endothelial receptors such as EPCR via PfEMP1 helps to evade clearance and can disrupt PC activation and APC cytoprotective signalling to contribute to vascular pathology. PfEMP1-EPCR interactions prevent PAR1 cytoprotective signalling by APC and can disrupt PC activation, suggesting a possible link between dysregulated PC pathway activity and endothelial dysfunction in cerebral malaria. Adjunctive therapies that could restore vascular dysfunction for cerebral malaria patients could potentially slow disease progression. The anti-inflammatory properties of APC make it an attractive therapeutic possibility in this context. Case studies have reported beneficial effects following recombinant APC infusion in patients with severe malaria. Signalling-selective APC variants with diminished anticoagulant properties could offer a safer approach to circumvent the increased bleeding risk associated with recombinant APC administration. In addition to the useful cytoprotective signalling properties of APC^{CIDR}, its ability to effectively compete with P. falciparum-infected erythrocytes for EPCR binding sites on the blood vessel surface, a feature unlikely to be achieved by other previously described APC variants already utilised in a clinical setting, further highlights its potential utility in this context.

In addition to its potential application as an adjunctive therapy for the treatment of cerebral malaria, APC^{CIDR} may possess haemostatic properties that could be utilised for the treatment of individuals with bleeding disorders. Uncontrolled bleeding remains a significant and unmet morbidity in several clinical settings, such as surgery, childbirth and traumatic injury. Major bleeding causes ˜40% of deaths associated with major trauma. Furthermore, PPH is the most common form of major obstetric haemorrhage and occurs in up to 18% of live births. Existing treatments to restore haemostasis in these settings are often ineffective and used without strong evidence to support their use. In addition, >30% of people with severe haemophilia receiving replacement therapy develop anti-FVIII antibodies that necessitate use of alternative haemostatic strategies to ‘bypass’ the inhibitory activity of anti-FVIII antibodies. Despite the recent development of novel non-factor bypass agents, these therapies have several limitations, including unclear mechanisms of action, potential risk of thrombosis and variable efficacy between patients.

Recent studies have demonstrated that incubation of the isolated CIDRα1.4 domain from PfEMP1 derived from the P. falciparum IT4var19 strain with primary human lung and dermal endothelial cells impedes PC activation by thrombin. Our data demonstrate that APC^{CIDR} also significantly inhibits EPCR-dependent activation of endogenous PC compared to inactivated wild type APC, which had little to no effect on PC activation even at the highest PC concentrations tested. This data suggests that attenuation of PC pathway activation may represent a novel potential mechanism for the treatment of both inherited and acquired bleeding disorders. Targeting and blocking natural anticoagulant pathways has become a prominent approach to restore haemostasis. Specifically, a mutant α1-antitrypsin SERPIN that specifically targets APC has been demonstrated to enhance thrombin generation in thrombin generation assays and limits bleeding in murine haemophilia models. Nevertheless, it is unclear, given the broad specificity of SERPINs for their substrates, whether this engineered SERPIN is sufficiently specific to not block the enzymatic activity of other related plasma proteases. Moreover, engineered SERPINs targeting APC would ultimately prevent APC cytoprotective activity. Another strategy to reduce endogenous APC anticoagulant activity for therapeutic benefit in individuals with uncontrolled bleeding was developed using an anti-APC monoclonal antibody that specifically blocks APC anticoagulant function. This antibody showed prophylactic efficacy in curbing bleeding in a haemophilia A monkey model, however, it also exhibited off-target inhibition of APC cytoprotective activities. Therefore, current therapies that successfully reduce APC anticoagulant activity to restore haemostasis 15 have to ‘trade-off’ attenuation of anticoagulant activity with some degree of loss of APC cytoprotective activity. In contrast, the Applicant proposes that APC^{CIDR} may represent an alternative strategy, in which APC anticoagulant activity is impaired by prolonged APC^{CIDR} occupancy of EPCR to limit APC generation, as observed in this study. Furthermore, EPCR occupancy by APC^{CIDR} and subsequent impairment of APC anticoagulant activity would not, unlike other APC-targeting therapies, be at the expense of APC cytoprotective signalling functions as the data presented here indicates these are entirely retained by APC^{CIDR}.

One of the most common co-morbidities associated with severe haemophilia A is haemophilic arthropathy, which commonly arises in people with haemophilia who suffer from frequent joint bleeds. Although the precise mechanism of haemophilic arthropathy development is poorly understood, iron deposition arising from haemolysis can induce inflammation and neo-angiogenesis to cause synovitis and destruction of articular cartilage in the joints of sufferers with this debilitating condition. Current treatment for haemophilic arthropathy is centred on re-dosing replacement FVIII to reduce bleeding risk and no specific therapies for haemophilic arthropathy currently exist. Interestingly, recent studies have demonstrated a deleterious effect of endogenous EPCR in the development of haemophilic arthropathy. Specifically, FVIII/mice with needle-induced joint bleeding develop haemophilic arthropathy similar to that observed in people with severe haemophilia. Mice deficient in both FVIII and EPCR, however, failed to develop significant arthropathy. Moreover, administration of anti-EPCR antibodies that block (A) PC binding protected FVIII−/− mice from development of bleeding-induced haemoarthrosis. These data indicate that impairment of PC-EPCR interactions may have potential as a target for new therapies for this condition. Within this context, the ability of APC^{CIDR} to bind EPCR with antibody-like affinity, while still enabling APC cytoprotective signalling via PAR1, suggests its potential application as a novel therapeutic avenue for this condition. Establishing the efficacy of APC^{CIDR} compared to existing commercially available pro-haemostatic therapeutic agents, in promoting bleeding cessation in pre-clinical bleeding models using haemophilic mice, and determination of its potential therapeutic activity in ameliorating haemophilic arthropathy, represent obvious next steps in translating the data generated in this chapter into tangible therapeutic outputs.

Therefore, although recombinant variants of APC have been generated previously that exhibit distinct functional properties to that of wild type APC. This invention, however, describes a unique conjugate that consists of fusion partners not previously generated together (for example, a truncated APC molecule fused to a PfEMP1 CIDR domain, or Factor VIIa fused to a PfEMP1 CIDR domain, or a meizothrombin molecule fused to the PfEMP1 CIDR domain). In addition to the unique composition of APC^{CIDR}, the fusion protein also possesses unique functional properties, specifically:
1. APC^{CIDR} has no anticoagulant activity;
2. Binds EPCR with up to 100-fold enhanced affinity compared to wild type APC;
3. Mediates cytoprotective and anti-inflammatory signalling pathways that are associated with enhanced wound healing and anti-inflammatory cellular responses at least as well as wild type APC, despite loss of anticoagulant activity;
4. Once EPCR bound, APC^{CIDR} induces similar or enhanced anti-inflammatory signalling compared to wild type APC; and
5. Acts as an adjunctive therapy for severe malaria as APC^{CIDR} competes with infected red blood cells for binding to EPCR to the vasculature, limiting cytoadhesion and promoting clearance of the infected red blood cells in the spleen.

The invention is therefore providing the first description of an APC-based enzyme, a meizothrombin-based enzyme and Factor VIIa-based enzyme with this unique combination of functional properties.

The invention may be used to treat individuals with, or at risk of, uncontrolled bleeding. Furthermore, it may be used to prevent or treat bleeding in individuals with other inherited bleeding disorders (including, but not limited, to factor XI deficiency, factor V deficiency, factor FVII deficiency and factor X deficiency, and also co-morbidities associated with haemophilia A, such as haemophilic arthropathy). The invention could also be used as an emergency haemostatic agent that prevents bleeding following trauma or surgery, or to reverse anticoagulant therapy.

This invention may be used to treat individuals with P. falciparum malaria, and other acute inflammatory disorders. APC^{CIDR} binds with up to 100-fold higher affinity to EPCR than its natural ligands and would therefore effectively compete for EPCR binding during malarial infection with P. falciparum-infected erythrocytes that use EPCR binding to persist and damage the cerebral vasculature. Consequently, APC^{CIDR} would attenuate malarial symptoms in P. falciparum-infected individuals being treated with anti-parasitic drugs that take up to 24 hours before becoming effective. Furthermore, once EPCR bound, APC^{CIDR} induces similar or enhanced anti-inflammatory signalling to wild type APC, suggesting the normal functioning of this pathway would not be compromised.

In the specification the terms “comprise, comprises, comprised and comprising” or any variation thereof and the terms “include, includes, included and including” or any variation thereof are considered to be totally interchangeable, and they should all be afforded the widest possible interpretation and vice versa.

The invention is not limited to the embodiments hereinbefore described but may be varied in both construction and detail.

1. A conjugate comprising a P. falciparum erythrocyte membrane protein 1 (PfEMP1) CIDR^{α1.4} domain defined by SEQ ID NO. 1, or a functional variant thereof, fused to a therapeutic agent.

2. The conjugate of claim 1, wherein the therapeutic agent is selected from an enzyme, an antibody, a small molecule inhibitor, a protein, and a drug.

3. The conjugate of claim 1 or claim 2, wherein the conjugate is an isolated recombinant fusion protein comprising a truncated APC molecule defined by SEQ ID NO. 6 fused to the PfEMP1 CIDR^{α1.4} domain defined by SEQ ID NO. 1, or functional variant thereof.

4. The conjugate of claim 1 or claim 2, wherein the conjugate is an isolated recombinant fusion protein comprising a Factor VIIa molecule defined by SEQ ID NO. 9 fused to the PfEMP1 CIDR^{α1.4} domain defined by SEQ ID NO. 1, or a functional variant thereof.

5. The conjugate of claim 1 or claim 2, wherein the conjugate is an isolated recombinant fusion protein comprising a meizothrombin molecule defined by SEQ ID NO. 13 fused to the PfEMP1 CIDR^{α1.4} domain defined by SEQ ID NO. 1, or functional variant thereof.

6. A conjugate comprising a P. falciparum erythrocyte membrane protein 1 (PfEMP1) CIDR^{α1.4} domain defined by SEQ ID NO. 1, or a functional variant thereof, fused to a therapeutic agent selected from a truncated APC molecule defined by SEQ ID NO. 6, a truncated Factor VIIa molecule defined by SEQ ID NO. 9, and a meizothrombin molecule defined by SEQ ID NO. 13.

7. The conjugate according to claim 6, wherein the therapeutic agent is a truncated APC molecule defined by SEQ ID NO. 6.

8. The conjugate according to claim 6, wherein the therapeutic agent is a truncated Factor VIIa molecule defined by SEQ ID NO. 9.

9. The conjugate according to claim 6, wherein the therapeutic agent is a truncated meizothrombin molecule defined by SEQ ID NO. 13.

10. A conjugate comprising a P. falciparum erythrocyte membrane protein 1 (PfEMP1) CIDR^{α1.4} domain defined by SEQ ID NO. 1, or a functional variant thereof, fused to a therapeutic agent for endothelial adhesion.

11. The conjugate of any one of claims 1 to 10 for use as a medicament.

12. The conjugate of claim 11 for use in the treatment of vascular dysfunction.

13. The conjugate of claim 12, wherein the vascular dysfunction is in subjects with an inflammatory disease or a thrombotic disease.

14. The conjugate of claim 13 for use in the treatment or prevention of inflammatory disease of claim 13, wherein the inflammatory disease is selected from diabetes, cardiovascular disease, arthritis, allergies, asthma, chronic obstructive pulmonary disease (COPD), psoriasis, acne, vasculitis, inflammatory bowel disease, multiple sclerosis, chronic inflammatory demyelinating polyneuropathy, Guillain-Barre syndrome, Grave's disease, myasthenia gravis, cerebral malaria, cancer, celiac disease, glomerulonephritis, hepatitis, cryopyrinopathies or cryopyrin-associated periodic syndromes (CAPS), disease caused by rhinoviruses, and coronaviruses.

15. The conjugate of claim 13 for use in the treatment or prevention of the thrombotic disease of claim 13, wherein the thrombotic disease is selected from deep vein thrombosis (DVT), ischemic stroke, Paget-Schroetter disease, Budd-Chiara syndrome, portal vein thrombosis, renal vein thrombosis, cerebral venous sincus thrombosis, jugular vein thrombosis, cavernous sinus thrombosis, arterial thrombosis, myocardial infarction, limb ischemia, hepatic artery thrombosis, and thrombotic thrombocytopenia purpura (TTP).

16. The conjugate of claim 11 for use in the treatment or prevention of a hemostatic disorder.

17. The conjugate of claim 16 for use in the treatment or prevention of the hemostatic disorder of claim 16, wherein the hemostatic disorder is selected from the group consisting of hemophilia A, hemophilia B, FVII deficiency, FV deficiency, FX deficiency, FXI deficiency, Glanzmann's thrombasthenia, Bernard-Soulier syndrome, von Willebrand diseases, hemophilic arthropathy, bleeding of unknown cause, menorrhagia, rare inherited platelet function disorders, bleeding associated with trauma, injury, thrombosis, thrombocytopenia, stroke, coagulopathy, disseminated intravascular coagulation (DIC) and over-anticoagulation treatment disorders.

18. A pharmaceutical composition comprising the conjugate according to any one of claims 1 to 10 and a biologically acceptable carrier.

19. An isolated recombinant fusion protein according to any one of claims 3 to 5 for use in a method of treating or preventing a hemostatic disorder.

20. An isolated recombinant fusion protein according to any one of claims 3 to 5 for use in a method of treating or preventing a vascular dysfunction in subjects with acute inflammatory diseases.

21. A nucleic acid encoding the recombinant fusion protein as defined by SEQ ID NO. 15, is encoded by SEQ ID NO. 16, or a variant thereof encoding a functional variant.

22. An expression vector comprising the nucleic acid of claim 21.

23. The expression vector of claim 22 selected from the group consisting of an adenovirus-associated virus (AAV) vector, a retroviral vector, an adenoviral vector, a plasmid, or a lentiviral vector. Preferably, said AAV vector comprises an AAV1, AAV2, AAV3, AAV4, AAV5, AAV6, AAV7, AAV8, AAV9, AAV10, AAVII, RhIO, Rh74 or AAV-218 AAV serotype.
